(19) United States
(12) Patent Application Publication
(10) Pub. No.: US 2025/0324933 A1
(43) Pub. Date: Oct. 23, 2025

(54) Crown Rot Resistance

(71) Applicant: Commonwealth Scientific and Industrial Research Organisation, Acton, ACT (AU)

(72) Inventors: Yunfeng Jiang, Chengdu, Sichuan (CN); Zhi Zheng, Acton, ACT (AU); Michael Ayliffe, Acton, ACT (AU); Donald Gardiner, St. Lucia, Queensland (AU); Chunji Liu, Acton, ACT (AU); Donna Glassop, Acton, ACT (AU)

(21) Appl. No.: 18/685,782

(22) PCT Filed: Aug. 22, 2022
(86) PCT No.: PCT/AU2022/050946

§ 371 Date: Feb. 22, 2024

(30) Foreign Application Priority Data
Aug. 23, 2021 (AU) 2021902650

(51) Int. Cl. A01H 1/00; A01H 1/04; C12N 9/02; C12Q 1/26

(52) U.S. Cl. CPC A01H 1/1255; A01H 1/04; C12N 9/0008; C12Q 1/26; C12Y 102/01044; G01N 2333/90203

(57) ABSTRACT

The present invention relates to a genetically modified plant which has enhanced resistance to one or more fungal pathogen(s).

### CROSS-REFERENCE TO RELATED APPLICATIONS

This application is a U.S. National Stage Application of PCT Application No. PCT/AU2022/050946 filed Aug. 22, 2022, which application claims priority to Australian Application No. 2021902650 filed Aug. 23, 2021, the disclosures of which applications are incorporated by reference in its entirety.

### FIELD OF THE INVENTION

The present invention relates to a genetically modified plant which has enhanced resistance to one or more fungal pathogen(s).

### REFERENCE TO AN ELECTRONIC SEQUENCE LISTING

The contents of the electronic sequence listing (RICE-238_SEQ_LIST.xml; Size: 97,139 bytes; and Date of Creation: Oct. 14, 2024) is herein incorporated by reference in its entirety.

### BACKGROUND OF THE INVENTION

Fusarium crown rot (FCR) is a chronic and severe disease affecting cereal production in semi-arid regions worldwide. It is caused by multiple species of Fusarium (including F. culmorum, F. avenaceum, F. poae and F. pseudograminearum) which are fungal pathogens. The pathogen can infect cereal crops early, resulting in seedling death prior to and after emergence. In older plants the disease can cause significant browning of subcrown internodes and leaf sheaths and the development of white heads with no or shriveled grains (Smiley et al., 2005; Chakraborty et al., 2006).

Reports show that FCR can reduce grain yield by up to 35% in the USA (Smiley et al., 2005), 43% in Turkey (Tunali et al., 2008) and 45% in Iran (Saremi et al., 2007). In Australia FCR is estimated to routinely cause up to 10% reduction in wheat grain yield, valued at approximately $88M dollars and has the potential to cause over $400M losses (Kazan and Gardiner, 2017). Agronomic practices and environmental factors influence the level of disease and the losses in any one growing season. It has long been recognized that growing FCR resistant varieties is a major component in minimizing FCR damage (Liu and Ogbonnaya, 2015). However, cereal varieties characterised by high levels of resistance to FCR are still not available and resistance not understood.

Several QTL conferring FCR resistance have been reported in barley (Liu and Ogbonnaya, 2015). Of them, the locus on 4HL (Qcrs.cpi-4H) has the largest effect and it explains up to 45% of the phenotypic variance (Chen et al., 2013). Ten pairs of NILs targeting this locus were developed. The presence of the resistance allele among the NILs reduced FCR severity by 44% on average (Habib et al., 2016). Data from multiple field trials show that the presence of the resistant allele at this locus can reduce yield loss due to FCR infection by more than 10% (Zheng et al., 2021). However the causative gene underlying the resistance is still unknown.

There is a need for genetically modified plants with enhanced resistance to fungal diseases such as crown rot.

### SUMMARY OF THE INVENTION

The present inventors have identified polypeptides which confer enhanced resistance biotrophic fungal pathogen(s) such as Fusarium sp.

Thus, in a first aspect the present invention provides a plant having a genetically modified gene encoding an atypical cinnamoyl-CoA dehydrogenase 2 (CAD2) polypeptide, wherein when expressed in the plant the polypeptide confers enhanced resistance to one or more biotrophic fungal pathogen(s) when compared to a corresponding plant lacking the gene.

In an embodiment, the genetically modified gene is an exogenous polynucleotide encoding the polypeptide. In an embodiment, the polynucleotide is operably linked to a promoter capable of directing expression of the polynucleotide in a cell of the plant. In an embodiment, the promoter directs gene expression in a leaf and/or stem cell.

In an embodiment, the one or more fungal pathogen(s) is a rot, rust or a mildew.

In an embodiment, the rot is crown rot.

In an embodiment, the one or more fungal pathogen(s) is a Fusarium sp. In an embodiment, the Fusarium sp. is Fusarium pseudograminearum, Fusarium oxysporum, Fusarium avenaceum, Fusarium culmorum, Fusarium graminearum or Fusarium poae.

In an embodiment, the Fusarium sp. is Fusarium pseudograminearum.

In an embodiment, the polypeptide is encoded by a polynucleotide which comprises nucleotides having a sequence as provided in any one of SEQ ID NO's 11 to 19, a sequence which is at least 40% identical to one or more of SEQ ID NO's 11 to 19, or a sequence which hybridizes to one or more of SEQ ID NO's 11 to 19.

In an embodiment, the polypeptide is encoded by a polynucleotide which comprises nucleotides having a sequence as provided in any one of SEQ ID NO's 11 to 19, 87 and 88, a sequence which is at least 40% identical to one or more of SEQ ID NO's 11 to 19, 87 and 88, or a sequence which hybridizes to one or more of SEQ ID NO's 11 to 19, 87 and 88.

In an embodiment, the plant is a cereal plant. Examples include, but are not limited to wheat, oats, rye, barley, rice, sorghum and maize. In an embodiment, the cereal plant is a barley plant.

In an embodiment, the plant is a legume plant. In an embodiment, the legume plant is soybean.

In an embodiment, the plant comprises one or more further genetic modifications encoding another plant pathogen resistance polypeptide. Examples of such other plant pathogen resistance polypeptides include, but are not limited to, Lr34, Lr1, Lr3, Lr2a, Lr3ka, Lr11, Lr13, Lr16, Lr17, Lr18, Lr21, LrB, Lr67, Lr46, Sr50, Sr33, Sr13, Sr26, Sr61, Sr2 and Sr35. In an embodiment, the plant further comprises Lr34, Lr67 and Lr46. In an embodiment, the plant further comprises Lr67.

In an embodiment, the plant is homozygous for one or more or all of the genetic modification(s).

In an embodiment, the plant is growing in a field.

In another aspect, the present invention provides a population of at least 100 plants of the invention growing in a field.

In another aspect, the present invention provides a process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence at least 40% identical to the amino acid sequence of any one or more of SEQ ID NO's 1 to 10,
ii) introducing the polynucleotide into a plant,
iii) determining whether the level of resistance to one or more fungal pathogen(s) is increased relative to a corresponding plant lacking the polynucleotide, and
iv) optionally, selecting a polynucleotide which when expressed produces a polypeptide which confers enhanced resistance to one or more fungal pathogen(s).

In another aspect, the present invention provides a process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence at least 40% identical to the amino acid sequence of any one or more of SEQ ID NO's 1 to 10, 82 and 83,
ii) introducing the polynucleotide into a plant,
iii) determining whether the level of resistance to one or more fungal pathogen(s) is increased relative to a corresponding plant lacking the polynucleotide, and
iv) optionally, selecting a polynucleotide which when expressed produces a polypeptide which confers enhanced resistance to one or more fungal pathogen(s).

In another aspect, the present invention provides a process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence at least 40% identical to the amino acid sequence of any one or more of SEQ ID NO's 1 to 10, and 79 to 83,
ii) introducing the polynucleotide into a plant,
iii) determining whether the level of resistance to one or more fungal pathogen(s) is increased relative to a corresponding plant lacking the polynucleotide, and
iv) optionally, selecting a polynucleotide which when expressed produces a polypeptide which confers enhanced resistance to one or more fungal pathogen(s).

In an embodiment, the polypeptide comprises amino acids having a sequence which is at least 90% identical to one or more of SEQ ID NO's 1 to 10.

In an embodiment, the polynucleotide comprises a sequence which is at least 90% identical to one or more of SEQ ID NO's 11 to 19, 82 and 83.

In an embodiment, the polynucleotide comprises a sequence which is at least 90% identical to one or more of SEQ ID NO's 11 to 19 and 79 to 83.

In an embodiment, the plant is a cereal plant or a legume plant.

In an embodiment, step ii) further comprises stably integrating the polynucleotide operably linked to a promoter into the genome of the plant.

In another aspect, the present invention provides a substantially purified and/or recombinant polypeptide which confers enhanced resistance to one or more fungal pathogen(s), wherein the polypeptide comprises amino acids having a sequence at least 40% identical to the amino acid sequence of any one or more of SEQ ID NO's 1 to 10.

In another aspect, the present invention provides a substantially purified and/or recombinant polypeptide which confers enhanced resistance to one or more fungal pathogen(s), wherein the polypeptide comprises amino acids having a sequence at least 40% identical to the amino acid sequence of any one or more of SEQ ID NO's 1 to 10, 82 and 83.

In another aspect, the present invention provides a substantially purified and/or recombinant polypeptide which confers enhanced resistance to one or more fungal pathogen(s), wherein the polypeptide comprises amino acids having a sequence at least 40% identical to the amino acid sequence of any one or more of SEQ ID NO's 1 to 10 and 79 to 83.

In an embodiment, the polypeptide comprises amino acids having a sequence which are at least 70% identical, at least 80% identical, at least 90% identical, or at least 95% identical, to SEQ ID NO:1.

In another aspect the present invention provides an isolated and/or exogenous polynucleotide encoding a polypeptide of the invention.

Also provided is a chimeric vector comprising a polynucleotide of the invention. In an embodiment, the polynucleotide is operably linked to a promoter.

In an embodiment, the vector comprises one or more further exogenous polynucleotides encoding another plant pathogen resistance polypeptide.

In another aspect, the present invention provides a recombinant cell comprising an exogenous polynucleotide of the invention, and/or a vector of the invention.

In an embodiment, the cell is a cereal plant cell or a legume plant cell.

In another aspect, the present invention provides a method of producing the polypeptide of the invention, the method comprising expressing in a cell or cell free expression system the polynucleotide of the invention.

In a further aspect, the present invention provides a transgenic non-human organism comprising an exogenous polynucleotide of the invention, a vector of the invention and/or a recombinant cell of the invention. In an embodiment, the transgenic non-human organism is a transgenic plant.

In a further aspect, the present invention provides a method of producing a cell of the invention, the method comprising the step of introducing the polynucleotide of the invention, or a vector of the invention, into a cell.

In another aspect, the present invention provides a method of producing a plant with a genetic modification(s) of the invention, the method comprising the steps of
i) introducing a genetic modification(s) to a plant cell such that the cell is capable of producing an atypical cinnamoyl-CoA dehydrogenase 2 (CAD2) polypeptide that confers upon the plant comprising the cell enhanced resistance to one or more biotrophic fungal pathogen(s) when compared to a corresponding plant lacking the genetic modification(s),
ii) regenerating a plant with the genetic modification(s) from the cell, and iii) optionally harvesting seed from the plant, and/or
iv) optionally producing one or more progeny plants from the genetically modified plants, thereby producing the plant.

In an embodiment, step i) comprises introducing a polynucleotide of the invention and/or a vector of the invention into the plant cell.

In another aspect, the present invention provides a method of producing a plant with a genetic modification(s) of the invention, the method comprising the steps of
i) crossing two parental plants, wherein at least one plant comprises a genetic modification(s) of the invention,
ii) screening one or more progeny plants from the cross in i) for the presence or absence of the genetic modification(s), and
iii) selecting a progeny plant which comprise the genetic modification(s), thereby producing the plant.

In an embodiment, step ii) comprises analysing a sample comprising DNA from the plant for the genetic modification(s).

In an embodiment, step iii) comprises
i) selecting progeny plants which are homozygous for the genetic modification(s), and/or
ii) analysing the plant or one or more progeny plants thereof for enhanced resistance to one or more fungal pathogen(s).

In an embodiment, the method further comprises
iv) backcrossing the progeny of the cross of step i) with plants of the same genotype as a first parent plant which lacked the genetic modification(s) for a sufficient number of times to produce a plant with a majority of the genotype of the first parent but comprising the genetic modification(s), and
v) selecting a progeny plant which has enhanced resistance to one or more fungal pathogen(s).

Also provided is a plant produced using a method of the invention.

Further, provided is the use of the polynucleotide of the invention, or a vector of the invention, to produce a recombinant cell and/or a transgenic plant.

In another aspect, the present invention provides a method for identifying a plant which has enhanced resistance to one or more fungal pathogen(s), the method comprising the steps of
i) obtaining a sample from a plant, and
ii) screening the sample for the presence or absence of an atypical cinnamoyl-CoA dehydrogenase 2 (CAD2) polypeptide which when expressed in the plant the polypeptide confers enhanced resistance to one or more biotrophic fungal pathogen(s) when compared to a corresponding plant lacking the gene, and/or screening the sample for the presence or absence of the polypeptide.

In an embodiment, the screening comprises amplifying a region of the genome of the plant.

In an embodiment, the method identifies a genetically modified plant of the invention.

Further, provided is a plant part of the plant of the invention. In an embodiment, the plant part is a seed that comprises the genetic modification(s).

In another aspect, the present invention provides a method of producing a plant part, the method comprising,
a) growing a plant of the invention, and
b) harvesting the plant part.

In an embodiment, the plant part is a seed.

In another aspect the present invention provides a method of producing flour, wholemeal, starch or other product obtained from seed, the method comprising;
a) obtaining seed of the invention, and
b) extracting the flour, wholemeal, starch or other product.

In another aspect the present invention provides a product produced from a plant of the invention and/or a plant part of the invention.

In an embodiment, the plant part is a seed.

In an embodiment, the product is a food product or beverage product. Examples include, but are not limited to, the food product being selected from the group consisting of: flour, starch, leavened or unleavened breads, pasta, noodles, animal fodder, animal feed, breakfast cereals, snack foods, cakes, malt, beer, pastries and foods containing flour-based sauces, or the beverage product being selected from beer or malt.

In an embodiment, the product is a non-food product.

In an aspect, the present invention provides a method of preparing a food product of the invention, the method comprising mixing seed, or flour, wholemeal or starch from the seed, with another food ingredient.

In an aspect, the present invention provides a method of preparing malt, comprising the step of germinating seed of the invention.

In an aspect, the present invention provides for the use of a plant of the invention, or part thereof, as animal feed, or to produce feed for animal consumption or food for human consumption.

In an aspect, the present invention provides for the use of a plant of the invention for controlling or limiting one or more fungal pathogen(s) in crop production.

In an aspect, the present invention provides a composition comprising one or more of a polypeptide of the invention, a polynucleotide of the invention, a vector of the invention, or a recombinant cell of the invention, and one or more acceptable carriers.

In an aspect, the present invention provides a method of identifying a compound that binds to a polypeptide comprising amino acids having a sequence as provided in any one of SEQ ID NO's 1 to 10, or an amino acid sequence which is at least 40% identical to any one or more of SEQ ID NO's 1 to 10, the method comprising:
i) contacting the polypeptide with a candidate compound, and
ii) determining whether the compound binds the polypeptide.

In an aspect, the present invention provides a method of identifying a compound that binds to a polypeptide comprising amino acids having a sequence as provided in any one of SEQ ID NO's 1 to 10, 82 and 83, or an amino acid sequence which is at least 40% identical to any one or more of SEQ ID NO's 1 to 10, 82 and 83, the method comprising:
i) contacting the polypeptide with a candidate compound, and
ii) determining whether the compound binds the polypeptide.

In an aspect, the present invention provides a method of identifying a compound that binds to a polypeptide comprising amino acids having a sequence as provided in any one of SEQ ID NO's 1 to 10 and 79 to 83, or an amino acid sequence which is at least 40% identical to any one or more of SEQ ID NO's 1 to 10 and 79 to 83, the method comprising:
i) contacting the polypeptide with a candidate compound, and
ii) determining whether the compound binds the polypeptide.

In an embodiment, the polypeptide comprises a sequence at least 90% identical to SEQ ID NO:1, and does not have one or more or all of;
i) a valine at a position corresponding to amino acid number 179 of SEQ ID NO:1,
ii) an isoleucine at a position corresponding to amino acid number 180 of SEQ ID NO:1,
iii) a valine at a position corresponding to amino acid number 181 of SEQ ID NO:1, and
iv) an asparagine at a position corresponding to amino acid number 182 of SEQ ID NO:1.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Fine-mapping based on a NIL-derived population placed the R gene in an interval containing 9 genes.

FIG. 2. Physical positions of the nine genes located in the targeted interval in WBR1 (R1) and Morex.

FIG. 3A. Predicted substrate binding sites between R & S alleles of CCAR in barley.

FIG. 3B. The predicted structure of HvCAD proteins from R1 (left) and Morex (right) generated from the structure of M. truncatula Mt-CAD2 (template 4qtz.1.A). The different residues in the substrate binding site (position 181) are marked.

FIG. 4. Alignment of the region surrounding the predicted substrate binding pocket of the Fusarium crown rot resistance allele (top row highlighted in yellow) to similar enzymes in other barley lines, cereals and other plants.

FIG. 5. FCR resistance of transgenic plants with (‘+’) or without (‘−’) the targeted gene.

FIG. 6. Comparison of amino acid sequences of the gene and its orthologs from different species.

FIG. 7. Target areas of selected gRNA's.

FIG. 8. A copy of the region with the putative substrate binding site alignment. Identified nucleotides indicate differences between the sequences.

FIG. 9. Amino acid sequence of the region around the substrate binding site. Amino acids shaded in black are the same for all sequences, grey shaded amino acids are different between sequences.

FIG. 10. gRNA's summarised for consideration aligned to all sequence homologues.

### KEY TO THE SEQUENCE LISTING

SEQ ID NO: 1—Amino acid sequence of barley CAD2 biotrophic fungal pathogen resistance polypeptide.

SEQ ID NO: 2—Amino acid sequence of barley CAD2 (susceptible) polypeptide.

SEQ ID NO: 3—Amino acid sequence of wheat CAD2 polypeptide encoded by chromosome 5 on the A genome.

SEQ ID NO: 4—Amino acid sequence of wheat CAD2 polypeptide encoded by chromosome 4 the D genome.

SEQ ID NO: 5—Amino acid sequence of wheat CAD2 polypeptide encoded by the B genome (allele 1).

SEQ ID NO: 6—Amino acid sequence of rice CAD2 polypeptide

SEQ ID NO: 7—Amino acid sequence of maize CAD2 polypeptide

SEQ ID NO: 8—Amino acid sequence of sorghum CAD2 polypeptide

SEQ ID NO: 9—Amino acid sequence of Medicago truncatula CAD2 polypeptide

SEQ ID NO: 10—Amino acid sequence of Brassica napus CAD2 polypeptide

SEQ ID NO: 11—Nucleotide sequence encoding barley CAD2 biotrophic fungal pathogen resistance polypeptide.

SEQ ID NO: 12—Nucleotide sequence encoding barley CAD2 (susceptible) polypeptide.

SEQ ID NO: 13—Nucleotide sequence encoding wheat CAD2 polypeptide encoded by chromosome 5 on the A genome.

SEQ ID NO: 14—Nucleotide sequence encoding wheat CAD2 polypeptide encoded by chromosome 4 the D genome.

SEQ ID NO: 15—Nucleotide sequence encoding wheat CAD2 polypeptide encoded by the B genome (allele 1).

SEQ ID NO: 16—Nucleotide sequence encoding rice CAD2 polypeptide.

SEQ ID NO: 17—Nucleotide sequence encoding maize CAD2 polypeptide.

SEQ ID NO: 18—Nucleotide sequence encoding sorghum CAD2 polypeptide.

SEQ ID NO: 19—Nucleotide sequence encoding Medicago truncatula CAD2 polypeptide.

SEQ ID NO's 20 to 51, 58, 59 and 71 to 78—Oligonucleotide primers.

SEQ ID NO's 52, 53 and 60 to 70—gRNA's.

SEQ ID NO: 79—Amino acid sequence of wheat CAD2 polypeptide encoded by chromosome 4 on the A genome (allele 1).

SEQ ID NO: 80—Amino acid sequence of wheat CAD2 polypeptide encoded by chromosome 4 on the A genome (allele 2).

SEQ ID NO: 81—Amino acid sequence of wheat CAD2 polypeptide encoded by chromosome 5 the D genome.

SEQ ID NO: 82—Amino acid sequence of wheat CAD2 polypeptide encoded by the B genome (allele 2).

SEQ ID NO: 83—Amino acid sequence of wheat CAD2 polypeptide encoded by the B genome (allele 3).

SEQ ID NO: 84—Nucleotide sequence encoding wheat CAD2 polypeptide encoded by chromosome 4 on the A genome (allele 1).

SEQ ID NO: 85—Nucleotide sequence encoding wheat CAD2 polypeptide encoded by chromosome 4 on the A genome (allele 2).

SEQ ID NO: 86—Nucleotide sequence encoding wheat CAD2 polypeptide encoded by chromosome 5 the D genome.

SEQ ID NO: 87—Nucleotide sequence encoding wheat CAD2 polypeptide encoded by the B genome (allele 2).

SEQ ID NO: 88—Nucleotide sequence encoding wheat CAD2 polypeptide encoded by the B genome (allele 3).

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, plant biotrophic fungal resistance, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

### Polypeptides

As used herein, the term “atypical cinnamoyl-CoA dehydrogenase polypeptide 2” or “CAD2” refer to a short-chain dehydrogenase/reductase (SDR) family (cd08958) (Pan et al., 2014). Examples of the CAD2 polypeptide family include polypeptides which share high primary amino acid sequence identity, for example at least 40%, at least 50%, at least 60%, at least 70%, least 80%, at least 90%, or at least 95% identity with the amino acid sequence of any one or more of SEQ ID NO's 1 to 10. In another embodiment, examples of the CAD2 polypeptide family include polypeptides which share high primary amino acid sequence identity, for example at least 40%, at least 50%, at least 60%, at least 70%, least 80%, at least 90%, or at least 95% identity with the amino acid sequence of any one or more of SEQ ID NO's 1 to 10, 82 and 83. In another embodiment, examples of the CAD2 polypeptide family include polypeptides which share high primary amino acid sequence identity, for example at least 40%, at least 50%, at least 60%, at least 70%, least 80%, at least 90%, or at least 95% identity with the amino acid sequence of any one or more of SEQ ID NO's 1 to 10, 79 to 83. The present inventors have determined that some variants of the CAD2 protein family, when expressed in a plant, confer upon the plant resistance to one or more biotrophic fungal pathogen(s) such as Fusarium sp. An example of such a variant comprises an amino acid sequence provided as SEQ ID NO:1. Thus, variants which confer resistance are referred to herein as CAD2 (resistant) polypeptides or proteins, whereas those which do not (see amino acid sequence provided as SEQ ID NO:2) are referred to herein as CAD2 (susceptible) polypeptides.

Polypeptides of the invention typically comprise a conserved 3D structure consisting of ‘Rossmann-fold’ β-sheet with α-helices on both sides, an N-terminal dinucleotide cofactor binding motif, and an active site with a catalytical residue motif YXXXK (Moummou et al., 2012). The Rossmann-fold NAD(p)H/NAD(p)(+) binding (NADB) domain. NAD binding involves H-bonding of residues in a turn between the first strand and the subsequent helix of the Rossmann-fold topology. Characteristically, this turn exhibits a consensus binding pattern similar to GXGXXG, in which the first 2 glycines participate in NAD(P)-binding, and the third facilitates close packing of the helix to the beta-strand. Typically, proteins in this family contain a second domain in addition to the NADB domain, which is responsible for specifically binding a substrate and catalyzing a particular enzymatic reaction.

Polypeptides of the invention typically have a TGXXGXX[GA]NADP-binding motif which glycine-rich region plays a critical role in domain stability. cofactor binding motif and a YXXXK active site motif, with the Tyr residue of the active site motif serving as a critical catalytic residue. In addition to the Tyr and Lys, with an upstream Ser and/or an Asn, contributing to the active site. The protein of the invention is proposed to belong to the SDR108E a large family whose members catalyze the reduction of several phenolic precursors 4-dihydroflavonol, anthocyanidin, cinnamoyl-CoA, phenylacetaldehyde or eutypine (Moummou et al., 2012). CAD2 utilize a reaction mechanism typical of classical SDRs, in which a Ser-Tyr-Lys catalytic triad mediates hydrogen-bonding crucial for activating the oxygen of the target carbonyl group and thereby promoting acceptance of a hydride transferred from the nicotinamide of NADPH (Pan et al., 2014). The amino acid residues involved in interactions with the NADPH cosubstrate are generally highly conserved among all SDR enzymes and in particular within the SDR108E/SDR115E family (Pan et al., 2014). Pan et al. (2014) suggests that CAD2 have other biological functions.

In an embodiment, the polypeptide comprises amino acids having a sequence at least 40% identical to the amino acid sequence of any one or more of SEQ ID NO's 1 to 10.

In an embodiment, the polypeptide comprises amino acids having a sequence at least 50% identical to the amino acid sequence of any one or more of SEQ ID NO's 1 to 10.

In an embodiment, the polypeptide comprises amino acids having a sequence at least 60% identical to the amino acid sequence of any one or more of SEQ ID NO's 1 to 10.

In an embodiment, the polypeptide comprises amino acids having a sequence at least 70% identical to the amino acid sequence of any one or more of SEQ ID NO's 1 to 10.

In an embodiment, the polypeptide comprises amino acids having a sequence at least 80% identical to the amino acid sequence of any one or more of SEQ ID NO's 1 to 10.

In an embodiment, the polypeptide comprises amino acids having a sequence at least 90% identical to the amino acid sequence of any one or more of SEQ ID NO's 1 to 10.

In an embodiment, the polypeptide comprises amino acids having a sequence at least 95% identical to the amino acid sequence of any one or more of SEQ ID NO's 1 to 10.

In an embodiment, the polypeptide comprises amino acids having a sequence at least 99% identical to the amino acid sequence of any one or more of SEQ ID NO's 1 to 10.

In an embodiment, the polypeptide comprises amino acids having a sequence at least 40% identical to the amino acid sequence of any one or more of SEQ ID NO's 1 to 10, 82 and 83.

In an embodiment, the polypeptide comprises amino acids having a sequence at least 50% identical to the amino acid sequence of any one or more of SEQ ID NO's 1 to 10, 82 and 83.

In an embodiment, the polypeptide comprises amino acids having a sequence at least 60% identical to the amino acid sequence of any one or more of SEQ ID NO's 1 to 10, 82 and 83.

In an embodiment, the polypeptide comprises amino acids having a sequence at least 70% identical to the amino acid sequence of any one or more of SEQ ID NO's 1 to 10, 82 and 83.

In an embodiment, the polypeptide comprises amino acids having a sequence at least 80% identical to the amino acid sequence of any one or more of SEQ ID NO's 1 to 10, 82 and 83.

In an embodiment, the polypeptide comprises amino acids having a sequence at least 90% identical to the amino acid sequence of any one or more of SEQ ID NO's 1 to 10, 82 and 83.

In an embodiment, the polypeptide comprises amino acids having a sequence at least 95% identical to the amino acid sequence of any one or more of SEQ ID NO's 1 to 10, 82 and 83.

In an embodiment, the polypeptide comprises amino acids having a sequence at least 99% identical to the amino acid sequence of any one or more of SEQ ID NO's 1 to 10, 82 and 83.

In an embodiment, the polypeptide comprises amino acids having a sequence at least 40% identical to the amino acid sequence of any one or more of SEQ ID NO's 1 to 10 and 79 to 83.

In an embodiment, the polypeptide comprises amino acids having a sequence at least 50% identical to the amino acid sequence of any one or more of SEQ ID NO's 1 to 10 and 79 to 83.

In an embodiment, the polypeptide comprises amino acids having a sequence at least 60% identical to the amino acid sequence of any one or more of SEQ ID NO's 1 to 10 and 79 to 83.

In an embodiment, the polypeptide comprises amino acids having a sequence at least 70% identical to the amino acid sequence of any one or more of SEQ ID NO's 1 to 10 and 79 to 83.

In an embodiment, the polypeptide comprises amino acids having a sequence at least 80% identical to the amino acid sequence of any one or more of SEQ ID NO's 1 to 10 and 79 to 83.

In an embodiment, the polypeptide comprises amino acids having a sequence at least 90% identical to the amino acid sequence of any one or more of SEQ ID NO's 1 to 10 and 79 to 83.

In an embodiment, the polypeptide comprises amino acids having a sequence at least 95% identical to the amino acid sequence of any one or more of SEQ ID NO's 1 to 10 and 79 to 83.

In an embodiment, the polypeptide comprises amino acids having a sequence at least 99% identical to the amino acid sequence of any one or more of SEQ ID NO's 1 to 10 and 79 to 83.

In an embodiment, the polypeptide comprises amino acids having a sequence at least 40% identical to SEQ ID NO:1.

In an embodiment, the polypeptide comprises amino acids having a sequence at least 50% identical to SEQ ID NO:1.

In an embodiment, the polypeptide comprises amino acids having a sequence at least 60% identical to SEQ ID NO:1.

In an embodiment, the polypeptide comprises amino acids having a sequence at least 70% identical to SEQ ID NO:1.

In an embodiment, the polypeptide comprises amino acids having a sequence at least 80% identical to SEQ ID NO:1.

In an embodiment, the polypeptide comprises amino acids having a sequence at least 90% identical to SEQ ID NO:1.

In an embodiment, the polypeptide comprises amino acids having a sequence at least 95% identical to SEQ ID NO:1.

In an embodiment, the polypeptide comprises amino acids having a sequence at least 99% identical to SEQ ID NO:1.

In an embodiment, the polypeptide comprises amino acids having a sequence as provided in SEQ ID NO:1.

In an embodiment, the polypeptide has one or more or all of;
i) an alanine at a position corresponding to amino acid number 179 of SEQ ID NO:1,
ii) a leucine at a position corresponding to amino acid number 180 of SEQ ID NO:1,
iii) a phenylalanine at a position corresponding to amino acid number 181 of SEQ ID NO:1, and
iv) a threonine at a position corresponding to amino acid number 182 of SEQ ID NO:1.

In an embodiment, the polypeptide has a phenylalanine at a position corresponding to amino acid number 181 of SEQ ID NO:1.

In an embodiment, the polypeptide has an alanine at a position corresponding to amino acid number 179 of SEQ ID NO:1 and/or a threonine at a position corresponding to amino acid number 182 of SEQ ID NO:1.

In an embodiment, the polypeptide does not have one or more or all of; i) a valine at a position corresponding to amino acid number 179 of SEQ ID NO:1,
ii) an isoleucine at a position corresponding to amino acid number 180 of SEQ ID NO:1,
iii) a valine at a position corresponding to amino acid number 181 of SEQ ID NO:1, and
iv) an asparagine at a position corresponding to amino acid number 182 of SEQ ID NO:1.

In an embodiment, the polypeptide does not have a valine at a position corresponding to amino acid number 179 of SEQ ID NO:1 and/or an asparagine at a position corresponding to amino acid number 182 of SEQ ID NO:1.

In an embodiment, the gene does not encode a polypeptide comprising amino acids having a sequence of any one of SEQ ID NO's 2 to 10. In an embodiment, the gene does not encode a polypeptide comprising amino acids having a sequence of any one of SEQ ID NO's 2 to 10 or 79 to 83.

As used herein, “resistance” is a relative term in that the presence of a polypeptide of the invention (i) reduces the disease symptoms of a plant comprising the gene (R (resistant) gene) that confers resistance, relative to a plant lacking the R gene, and/or (ii) reduces pathogen reproduction or spread on a plant or within a population of plants comprising the R gene. Resistance as used herein is relative to the “susceptible” response of a plant to the same pathogen. Typically, the presence of the R gene improves at least one production trait of a plant comprising the R gene when infected with the pathogen, such as grain yield, when compared to an isogenic plant infected with the pathogen but lacking the R gene. The isogenic plant may have some level of resistance to the pathogen, or may be classified as susceptible. Thus, the terms “resistance” and “enhanced resistance” are generally used herein interchangeably.

Furthermore, a polypeptide of the invention does not necessarily confer complete pathogen resistance, for example when some symptoms still occur or there is some pathogen reproduction on infection but at a reduced amount within a plant or a population of plants. Resistance may occur at only some stages of growth of the plant, for example in adult plants (fully grown in size) and less so, or not at all, in seedlings, or at all stages of plant growth. In an embodiment, resistance occurs at the adult and the seedling stage. In an embodiment, resistance occurs at the adult stage. By using a transgenic strategy to express an CAD2 polypeptide in a plant, the plant of the invention can be provided with resistance throughout its growth and development. Enhanced resistance can be determined by a number of methods known in the art such as analysing the plants for the amount of pathogen and/or analysing plant growth or the amount of damage or disease symptoms to a plant in the presence of the pathogen, and comparing one or more of these parameters to an isogenic plant lacking an exogenous gene encoding a polypeptide of the invention.

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 90% free from other components with which it is naturally associated. In an embodiment, the polypeptide of the invention has an amino acid sequence which is different to a naturally occurring CAD2 polypeptide i.e. is an amino acid sequence variant.

Transgenic organisms, such as plants, and host cells of the invention may comprise an exogenous polynucleotide encoding a polypeptide of the invention. In these instances, the plants and cells produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. Typically, the cell comprises a non-endogenous gene that causes an altered amount of the polypeptide to be produced. In an embodiment, a “recombinant polypeptide” is a polypeptide made by the expression of an exogenous (recombinant) polynucleotide in a plant cell.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 300 amino acids in length, and the GAP analysis aligns the two sequences over a region of at least 300 amino acids. More preferably, the query sequence is at least 325 amino acids in length and the GAP analysis aligns the two sequences over a region of at least 335 amino acids. Even more preferably, the query sequence is at least 350 amino acids in length and the GAP analysis aligns the two sequences over a region of at least 350 amino acids. Even more preferably, the GAP analysis aligns two sequences over their entire length.

In some embodiments, the polypeptide is a biologically active fragment. As used herein a “biologically active” fragment is a portion of a polypeptide of the invention which maintains a defined activity of the full-length polypeptide such as when expressed in a plant, such as barley, confers (enhanced) resistance one or more biotrophic fungal pathogen(s) such as Fusarium sp when compared to an isogenic plant not expressing the polypeptide. Biologically active fragments can be any size as long as they maintain the defined activity but are preferably at least 320 residues long. Preferably, the biologically active fragment maintains at least 10%, at least 50%, at least 75% or at least 90%, of the activity of the full length protein. Biologically active fragments can easily be identified by deleting some of the N-terminus and/or C-terminus of the polypeptide and analyse the fragment for conferring enhanced resistance as defined herein.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 50%, at least 60%, at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In an embodiment, a polypeptide of the invention, other than that with an amino acid sequence provided as SEQ ID NO:1, is not a naturally occurring polypeptide.

As used herein, the phrase “at a position corresponding to amino acid number” or variations thereof refers to the relative position of the amino acid compared to surrounding amino acids. In this regard, in some embodiments a polypeptide of the invention may have deletional or substitutional mutation which alters the relative positioning of the amino acid when aligned against, for instance, SEQ ID NO:1.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have one, two, three, four or less than 10 amino acid changes relative to the reference polypeptide such as comprising an amino acid provided in SEQ ID NO:1.

Mutant (altered) polypeptides can be prepared using any technique known in the art, for example, using directed evolution, rational design strategies or mutagenesis (see below). Products derived from mutated/altered DNA can readily be screened using techniques described herein to determine if, when expressed in a plant, such as barley, confer (enhanced) resistance to one or more biotrophic fungal pathogen(s) such as Fusarium sp. For instance, the method may comprise producing a transgenic plant expressing the mutated/altered DNA and determining the effect of the pathogen on the growth of the plant.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 1 under the heading of “exemplary substitutions”.

In an embodiment, a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different polypeptides provided herewith, and/or not in the important motifs of CAD2 polypeptides identified herein. As the skilled person would be aware, such minor changes can reasonably be predicted not to alter the activity of the polypeptide when expressed in a recombinant cell.

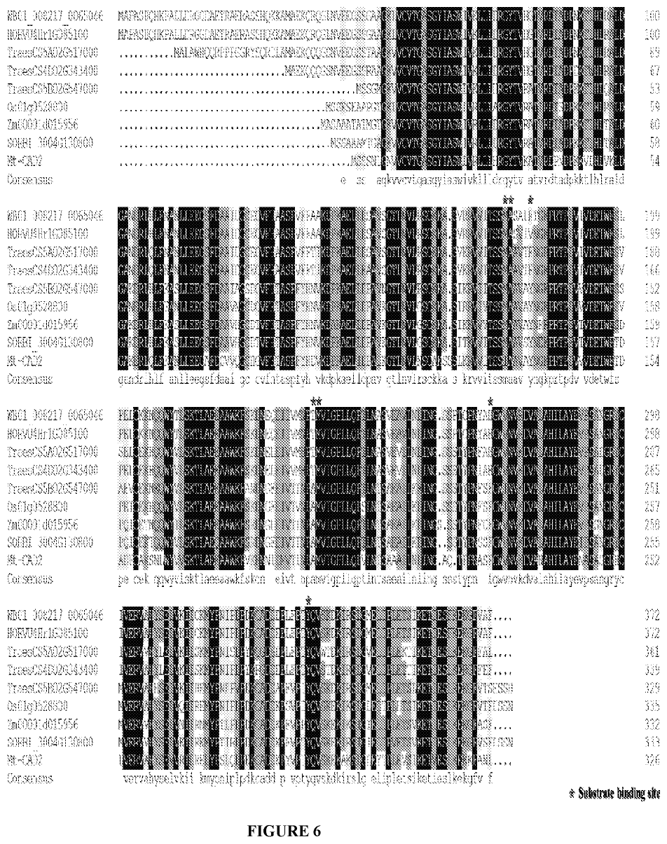

The primary amino acid sequence of a polypeptide of the invention can be used to design variants/mutants thereof based on comparisons with closely related polypeptides (for example, as shown in FIG. 6). As the skilled addressee will appreciate, residues highly conserved amongst closely related proteins are less likely to be able to be altered, especially with non-conservative substitutions, and activity maintained than less conserved residues (see above).

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. The polypeptides may be post-translationally modified in a cell, for example by phosphorylation, which may modulate its activity. These modifications may serve to increase the stability and/or bioactivity of the polypeptide of the invention.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

### Directed Evolution

In directed evolution, random mutagenesis is applied to a protein, and a selection regime is used to pick out variants that have the desired qualities, for example, increased activity. Further rounds of mutation and selection are then applied. A typical directed evolution strategy involves three steps:
1) Diversification: The gene encoding the protein of interest is mutated and/or recombined at random to create a large library of gene variants. Variant gene libraries can be constructed through error prone PCR (see, for example, Leung, 1989; Cadwell and Joyce, 1992), from pools of DNaseI digested fragments prepared from parental templates (Stemmer, 1994a: Stemmer, 1994b; Crameri et al., 1998; Coco et al., 2001) from degenerate oligonucleoides (Ness et al., 2002, Coco, 2002) or from mixtures of both, or even from undigested parental templates (Zhao et al., 1998; Eggert et al., 2005; Jézéquek et al. 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).
2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a host organism or part thereof and assaying the level of activity.
3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interative Protein Redesign and Optimization), EGAD (A Genetic Algorithm for Protein Design), Rosetta Design, Sharpen, and Abalone.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. Less preferred polynucleotides include tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. The term “polynucleotide” is used interchangeably herein with the term “nucleic acid”. Preferred polynucleotides of the invention encode a polypeptide of the invention.

By “isolated polynucleotide” we mean a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

The present invention involves modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

A “CAD2 gene” as used herein refers to a nucleotide sequence which is homologous to an isolated CAD cDNA (such as provided in SEQ ID NO: 11, or one or more or all of SEQ ID NO's 11 to 19, or one or more or all of SEQ ID NO's 11 to 19 and 84 to 88). As described herein, some alleles and variants of the CAD2 gene family encode a protein that confers resistance to one or more biotrophic fungal pathogen(s) such as Fusarium sp. CAD2 genes include the naturally occurring alleles or variants existing in cereals such as barley, as well as artificially produced variants.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. As described herein, the barley CAD2 genes (both resistant and susceptible alleles) contain two introns in their protein coding regions. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region of an CAD2 gene is operably linked to a promoter or polyadenylation/terminator region which is heterologous to the CAD2 gene, thereby forming a chimeric gene. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

The term “genetically modified”, “genetic modification” or variants thereof refers to any genetic manipulation by man and includes introducing genes into cells by transformation or transduction, gene editing, mutating genes in cells and altering or modulating the regulation of a gene in a cell or organisms to which these acts have been done or their progeny and so on.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

In an embodiment, a polynucleotide of the invention does not encode a polypeptide comprising amino acids having a sequence of any one of SEQ ID NO's 2 to 10. In an embodiment, a polynucleotide of the invention does not encode a polypeptide comprising amino acids having a sequence of any one of SEQ ID NO's 2 to 10 and 79 to 83.

In an embodiment, the polynucleotide does not have a nucleotide sequence as shown in any one of SEQ ID NO's 12 to 19. In an embodiment, the polynucleotide does not have a nucleotide sequence as shown in any one of SEQ ID NO's 12 to 19 and 84 to 88.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences, or the reverse complement, provided as SEQ ID NO's 11 to 19, provided as SEQ ID NO's 11 to 19 and 85 to 88, such as SEQ ID NO: 11. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide or an oligonucleotide of the invention includes molecules of varying sizes of, and/or are capable of hybridising to, the barley genome close to that of the reference polynucleotide or oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close to, for example to within 50 nucleotides, the region of the plant genome where the specific oligonucleotides defined herein hybridise. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

### Nucleic Acid Constructs

The present invention includes nucleic acid constructs comprising the polynucleotides of the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in leaves and/or stems of a plant, preferably a cereal plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages such as adults or seedlings. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

In an embodiment, the promoter is a stem-specific promoter, a leaf-specific promoter or a promoter which directs gene expression in an aerial part of the plant (at least stems and leaves) (green tissue specific promoter) such as a ribulose-1,5-bisphosphate carboxylase oxygenase (RUBISCO) promoter.

Examples of stem-specific promoters include, but are not limited to those described in U.S. Pat. No. 5,625,136, and Bam et al. (2008).

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters, tissue specific promoters (see, e.g., U.S. Pat. No. 5,459,252 and WO 91/13992); promoters from viruses (including host specific viruses), or partially or wholly synthetic promoters. Numerous promoters that are functional in mono- and dicotyledonous plants are well known in the art (see, for example, Greve, 1983; Salomon et al., 1984; Garfinkel et al., 1983; Barker et al., 1983); including various promoters isolated from plants and viruses such as the cauliflower mosaic virus promoter (CaMV 35S, 19S). Non-limiting methods for assessing promoter activity are disclosed by Medberry et al. (1992, 1993), Sambrook et al. (1989, supra) and U.S. Pat. No. 5,164,316.

Alternatively, or additionally, the promoter may be an inducible promoter or a developmentally regulated promoter which is capable of driving expression of the introduced polynucleotide at an appropriate developmental stage of the, for example, plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “vector” or “chimeric vector” is meant a nucleic acid molecule, preferably a DNA molecule derived, for example, from a plasmid, bacteriophage, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (Niedz et al., 1995) or derivatives thereof; a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

The level of a protein of the invention may be modulated by increasing the level of expression of a nucleotide sequence that codes for the protein in a plant cell, or decreasing the level of expression of a gene encoding the protein in the plant, leading to modified pathogen resistance. The level of expression of a gene may be modulated by altering the copy number per cell, for example by introducing a synthetic genetic construct comprising the coding sequence and a transcriptional control element that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in a substantial modification of pathogen resistance or other phenotype. Alternatively, a population of mutagenized seed or a population of plants from a breeding program may be screened for individual lines with altered pathogen resistance or other phenotype associated with pathogen resistance.

### Recombinant Cells

Another embodiment of the present invention includes a recombinant cell comprising a host cell transformed with one or more recombinant molecules of the present invention, or progeny cells thereof. Transformation of a nucleic acid molecule into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing is used to transform the target cell using, for example, targeting nucleases such as TALEN, Cpf1 or Cas9-CRISPR or engineered nucleases derived therefrom.

A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a cereal plant, more preferably barley or wheat cells, and even more preferably a barley cell.

### Genome Editing

Endonucleases can be used to generate single strand or double strand breaks in genomic DNA. The genomic DNA breaks in eukaryotic cells are repaired using non-homologous end joining (NHEJ) or homology directed repair (HDR) pathways. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into three major groups using various nucleases or combinations on nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by the synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a U3 or U6 small nuclear RNA promoter. The sgRNA recognises the specific gene and part of the gene for targeting. The protospacer adjacent motif (PAM) is adjacent to the target site constraining the number of potential CRISPR-Cas targets in a genome although the expansion of nucleases also increases the number of PAM's available. There are numerous web tools available for designing gRNAs including CHOPCHOP (http://chopchop.cbu.uib.no), CRISPR design https://omictools.com/crispr-design-tool, E-CRISP http://www.e-crisp.org/E-CRISP/, Geneious or Benchling https://benchling.com/crispr. Examples of gRNA's that can be used in the inventioninclude those comprising a nucletode sequence provided in 52 to 57 and 60 to 70 (see Examples 6 and 7).

CRISPR-Cas systems are the most frequently adopted in eukaryotic work to date using a Cas9 effector protein typically using the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Due to the cell wall on plant cells the delivery of the CRISPR-Cas machinery into the cell and successful transgenic regenerations have used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, accession number KR029109.1).

Alternative CRISPR-Cas systems refer to effector enzymes that contain the nuclease RuvC domain but do not contain the HNH domain including Cas12 enzymes including Cas12a, Cas12b, Cas12f, Cpf1, C2c1, C2c3, and engineered derivatives. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c.

### Sequence Insertion or Integration

The CRISPR-Cas system can be combined with the provision of a nucleic acid sequence to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S.pyogenes Cas9 nuclease with a D10A mutation or H840A mutation.

### Genome Base Editing or Modification

Base editors have been created by fusing a deaminase with a Cas9 domain (WO 2018/086623). By fusing the deaminase can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of Cas9 ribonucleoproteins (RNPs) which are transformed into the cell or protoplast and does not rely on the Cas9 being integrated into the host genome, thereby reducing the undesirable side cuts that has been linked with the random integration of the Cas9 gene. Only short flanking sequences are required to form a stable Cas9 and sgRNA stable ribonucleoprotein in vitro. Woo et al. (2015) produced pre-assembled Cas9/sgRNA protein/RNA complexes and introduced them into protoplasts of Arabidopsis, rice, lettuce and tobacco and targeted mutagenesis frequencies of up to 45% observed in regenerated plants. RNP and in vitro demonstrated in several species including dicot plants (Woo et al., 2015), and monocots maize (Svitashev et al., 2016) and wheat (Liang et al., 2017). Genome editing of plants using CRISPR-Cas 9 in vitro transcripts or ribonucleoproteins are fully described in Liang et al. (2018) and Liang et al. (2019).

### Method for Gene Insertion

Plant embryos may be bombarded with a Cas9 gene and sgRNA gene targeting the site of integration along with the DNA repair template. DNA repair templates are may be synthesised DNA fragment or a 127-mer oligonucleotide, with each encoding the cDNA or the gene of interest. Bombarded cells are grown on tissue culture medium. DNA extracted from callus or TO plants leaf tissue using CTAB DNA extraction method can be analysed by PCR to confirm gene integration. T1 plants selected if pcr confirms presence of the gene of interest.

The method comprises introducing into a plant cell the DNA sequence of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site, the donor DNA is inserted into the transformed plant's genome.

Although homologous recombination occurs at low frequency in plant somatic cells the process appears to be increased/stimulated by the introduction of doublestrand breaks (DSBs) at selected endonuclease target sites. Ongoing efforts to generate Cas, in particular Cas9, variants or alternatives such as Cpf1 or Cmsl may improve the efficiency.

### Transgenic Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

A “transgenic plant” as used herein refers to a plant that contains a nucleic acid construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is preferably stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”.

A “non-transgenic plant” is one which has not been genetically modified by the introduction of genetic material by human intervention using, for example, recombinant DNA techniques. In a preferred embodiment, the transgenic plants are homozygous for each and every gene that has been introduced (transgene) so that their progeny do not segregate for the desired phenotype.

As used herein, the term “compared to an isogenic plant”, or similar phrases, refers to a plant which is isogenic, or is substantially isogenic” relative to the transgenic plant but without the transgene of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant. “Wild type” or “corresponding”, as used herein, refers to a cell, tissue or plant that has not been modified according to the invention. Wild-type or corresponding cells, tissue or plants may be used as controls to compare levels of expression of an exogenous nucleic acid or the extent and nature of trait modification with cells, tissue or plants modified as described herein.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. This would generally be to modulate the production of at least one protein defined herein in the desired plant or plant organ. Transgenic plant parts include all parts and cells of said plants comprising the transgene such as, for example, cultured tissues, callus and protoplasts.

Plants contemplated for use in the practice of the present invention include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plant is a cereal plant. In an embodiment, the cereal plant is wheat. In an embodiment, the cereal plant is rice. In an embodiment, the cereal plant is maize. In an embodiment, the cereal plant is triticale. In an embodiment, the cereal plant is oats. In an embodiment, the cereal plant is barley.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare or suitable for commercial production of grain.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

As used herein, the “other genetic markers” may be any molecules which are linked to a desired trait of a plant. Such markers are well known to those skilled in the art and include molecular markers linked to genes determining traits such disease resistance, yield, plant morphology, grain quality, dormancy traits, grain colour, gibberellic acid content in the seed, plant height, flour colour and the like. Examples of such genes are the rust resistance genes mentioned herein, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265).

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated/crossed to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of polynucleotides such as DNA into plants by direct transfer into pollen, by direct injection of polynucleotides such as DNA into reproductive organs of a plant, or by direct injection of polynucleotides such as DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. Nos. 5,569,834, 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. Nos. 5,589,617, 6,541,257, and other methods are set out in WO 99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Marker Assisted Selection

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis of at least one CAD2 allele or variant that confers upon the plant resistance to one or more biotrophic fungal pathogen(s) such as Fusarium sp, allows rapid selection of plants carrying the desired trait, which may be nurtured to maturity in the greenhouse or field for subsequent further backcrossing to the recurrent parent.

Any molecular biological technique known in the art can be used in the methods of the present invention. Such methods include, but are not limited to, the use of nucleic acid amplification, nucleic acid sequencing, nucleic acid hybridization with suitably labelled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of the (for example) CAD2 gene which confers upon the plant resistance to one or more biotrophic fungal pathogen(s) such as Fusarium sp. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al. (2001).

In an embodiment, a linked loci for marker assisted selection is at least within 1 cM, or 0.5 cM, or 0.1 cM, or 0.01 cM from a gene encoding a polypeptide of the invention.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G. Moller (editors), BIOS Scientific Publishers Ltd, Oxford, (2000)). PCR can be performed on cDNA obtained from reverse transcribing mRNA isolated from plant cells expressing a CAD2 gene or allele which confers upon the plant resistance to one or more biotrophic fungal pathogen(s) such as Fusarium sp. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

### TILLING

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Plant/Grain Processing

Grain/seed of the invention, preferably cereal grain and more preferably barley or wheat grain, or other plant parts of the invention, can be processed to produce a food ingredient, food or non-food product using any technique known in the art.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain such as wheat or barley grain. The particle size of refined flour is described as flour in which not less than 98% passes through a cloth having openings not larger than those of woven wire cloth designated “212 micrometers (U.S. Wire 70)”. The coarse fraction includes at least one of: bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e. -ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively, bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain such as barley and wheat grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:
(i) providing grain, such as barley or wheat grain, of the invention,
(ii) steeping said grain,
(iii) germinating the steeped grains under predetermined conditions and
(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of speciality malts, including, but limited to, methods of roasting the malt.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

### EXAMPLES

### Example 1—Materials and Methods

### Plant Materials

A NIL-derived population consisting of 2,203 lines was generated and used to further delineate the 13 markers co-segregating with the R locus Qcrs.cpi-4H at 4HL. The population was generated based on seven heterozygous plants identified with the SSR marker HVM67 (forward primer GTCGGGCTCCATTGCTCT (SEQ ID NO:20) and reverse primer CCGGTACCCAGTGACGAC (SEQ ID NO: 21)). This marker was among the markers closely linked to the R locus on 4HL identified in the initial detection of the locus (Chen et al., 2013), and it was thus used in developing NIL_CR4HL_1R/1S (NIL1) targeting this locus from the population of Baudin/CRCS237 (Habib et al., 2016).

The seven heterozygous plants (at F5 generation) were sown in pots and grown in glasshouses at Queensland Bioscience Precinct (QBP) at CSIRO St Lucia laboratories in Brisbane, Australia. About 3,000 seeds were harvested from the seven plants. The harvested seeds were germinated in Petri dishes on three layers of filter paper saturated with water. Seedlings of 3-day-old were planted into each 5 cm square punnet (Rite Grow Kwik Pots, Garden City Plastics, Australia) containing sterilized University of California mix C (50% sand and 50% peat v/v). The punnets were put into a glasshouse with the following settings: 25/18 (±1) ° C. day/night temperature and 65/80% (±5) % day/night relative humidity, with natural sunlight levels and variable photoperiod depending on the time of year. These plants were all self-pollinated and a single seed from each of the plants was harvested and grew for generating the next generation. Based on this method of single-seed descendent, the materials were processed to F10 generation. Seeds from 2,203 of these F10 lines were used in the map-based cloning study, and the numbers of seeds harvested from each of these F10 lines varied from 5 to 20.

### Identification of the Targeted Interval Containing the Gene Underlying FCR Resistance at the 4HL Locus Qcrs.Cpi-4H

### Dna Extraction

Two seeds from each of the 2,203 lines of the NIL-derived population were germinated in trays. DNA was extracted from each of these lines using fresh leaf tissue of five-day-old seedlings based on the CTAB method. Briefly, leaf tissue was broken down by grinding in the presence of liquid nitrogen. The CTAB extraction buffer (100 mM Tris-HCl pH 8.0, 1.4 M NaCl, 20 mM EDTA pH 8.0, 2.0% w/v CTAB, 1.0% w/v PVP, 0.2% v/v β-mercaptoethanol) was then added, and after incubation at 65° C., purification with phenol:chloroform:isoamyl alcohol (25:24:1) and precipitation with isopropanol were conducted. Finally, DNA was dissolved in 50 μl of pure water and DNA yield among the samples varied from 10 to 150 μg.

### Genotyping of the NIL-Derived Population and Identification of Key Recombinant Lines for the Targeted Region

The whole population was genotyped with 15 markers (Table 2) identified in an earlier study, two of them flanking the targeted locus (Morex_254670 and Morex-38190) and the other 13 co-segregated with the locus Qcrs.cpi-4H (Jiang et al., 2019).

PCR reactions were performed in Applied Biosystems® GeneAmp®m PCR System 2700 (Applied Biosystems Inc., Foster City, CA) in volumes of 10 μL containing 25 ng genomic DNA, 0.20 μM of each primers, 2 mM MgCl₂, 0.2 mM dNTP and 0.5 unit Taq DNA polymerase. The PCR conditions were as follows: 94° C. for 5 min, followed by 35 cycles of 94° C. for 30 s, 50-60° C. for 30 s (depending on primers, Table 2), 72° C. for 1 min and a final extension for 7 min at 72 TC. PCR products were then separated in 2% agarose gels.

Key recombinant lines for the targeted region were selected based on the profiles of the two markers flanking the targeted locus Qcrs.cpi-4H (Morex_254670 and Morex-38190). They were lines for which one of the markers showing the resistant allele from R1 and the other marker showing susceptible allele from Baudin. Seeds from each of these key recombinant lines were increased in pots in the QBP glasshouses at CSIRO St Lucia site. Levels of FCR resistance (phenotypes) for each of these key recombinant lines was determined by conducting ten independent experimental trials as described below.

TABLE 2
Primers for markers used for map-based gene cloning*

| | | | Annealing |
|---|---|---|---|
| Marker | Forward primer | Reverse primer | temperature |
| Morex_254670 | GCGATGTCTACAGTAGGAG | GCTTGAGCCATAATACCATT | 58 |
| | (SEQ ID NO: 22) | (SEQ ID NO: 23) | |
| Morex_5853-1 | CACTCCTCGCTTCTCTTC | GATTCACCATTGTCACTCTG | 60 |
| (EcoRV) | (SEQ ID NO: 24) | (SEQ ID NO: 25) | |
| Morex_5853-2 (PstI) | CATCAGACTCACCACCTTC | CGTAGCATTCACTTACTCCT | 60 |
| | (SEQ ID NO: 26) | (SEQ ID NO: 27) | |
| Morex_159900 | TCGGGTCAGGCTTTTTTT | GGTGTTCACGATAGTTAGG | 53 |
| | (SEQ ID NO: 28) | (SEQ ID NO: 29) | |
| Morex_60022 | TGATGGATGGCGTTGAATA | CTTAGGCTTGTTGGTTGTC | 60 |
| | (SEQ ID NO: 30) | (SEQ ID NO: 31) | |
| Morex_1571262 | CTCTAAGCACTTTGAATAAGGG | GCGTGTGTCTCTATAAGGAA | 58 |
| | (SEQ ID NO: 32) | (SEQ ID NO: 33) | |
| Morex_1580013 | ACGAATTGCTCAGTCAGAT | GCGGTCAGGTCAAGTTAA | 60 |
| (HindiIII) | (SEQ ID NO: 34) | (SEQ ID NO: 35) | |
| Morex_88749 (SalI) | CGGTTCCATCTTCTTCCAT | TCAAGGCATCAAGCATC | 60 |
| | (SEQ ID NO: 36) | (SEQ ID NO: 37) | |
| Morex_1572596 | TCTCCGAAGAGCATATACGA | GTGGGTGGTGGATTAGGT | 60 |
| | (SEQ ID NO: 38) | (SEQ ID NO: 39) | |
| Morex_244100 | CGACAATGTTCATCCGATT | GGTACAGTGTTGCGAGTT | 58 |
| | (SEQ ID NO: 40) | (SEQ ID NO: 41) | |
| Morex_1586696 | CACTAAGGAATCCGATGAAATC | CATGACCTGAAACGATGAATAG | 60 |
| | (SEQ ID NO: 42) | (SEQ ID NO: 43) | |
| Morex_52829 | CTCACTATTCAGCCAGACA | ATTGACAGGAGACATGACAT | 60 |
| | (SEQ ID NO: 44) | (SEQ ID NO: 45) | |
| Morex_59399 | ATCACTGCTGCTCAGATAG | TGCGTCTTCTTCACCATG | 60 |
| (Nael) | (SEQ ID NO: 46) | (SEQ ID NO: 47) | |
| Morex_130357 | GGTTTAGAATTTGTCCAGGT | GTTGGGCGTTTCAATAAATG | 60 |
| | (SEQ ID NO: 48) | (SEQ ID NO: 49) | |
| Morex_38190(SmaI) | ATATAAACCGTCCCTCCCT | CCATCGTTGATGATGAGTG | 57 |
| | (SEQ ID NO: 50) | (SEQ ID NO: 51) | |

*CAPS markers and the amplicons were digested with suitable restriction enzymes listed in brackets.

### Evaluation of FCR Resistance

### Inoculum Preparation

A highly aggressive strain of F. pseudograminearum (CS3096) was used in this study. It is a strain isolated from infected crowns of wheat in northern New South Wales, Australia (Mitter et al., 2006). Plates of ½ strength potato dextrose agar (PDA) inoculated with the F. pseudograminearum strain were incubated for 12 days at room temperature before the mycelium in the plate was scraped. The plates were then incubated for an additional 5-7 days under a combination of cool white and black (UVA) fluorescent lights with a 12-h photoperiod. The spores were then harvested, and the concentration of spore suspension was adjusted to meet experimental requirements. Tween 20 was added to the spore suspension to a final concentration of 0.1% v/v prior to use for inoculation.

### Inoculation of Barley Lines and FCR Assessment

FCR assessments were all conducted in the controlled environment facilities (CEF) of CSIRO St Lucia laboratories in Brisbane. Methods used for FCR inoculation and assessment were as described by Li et al. (2008). Briefly, seeds were germinated in Petri dishes on two layers of filter paper saturated with water. The germinated seedlings (4 days post-germination) were immersed in the spore suspension for 1 min. The 56-well plastic trays (Rite Grow Kwik Pots, Garden City Plastics, Australia) containing steam-sterilized University of California mix C (50% sand and 50% peat v/v) were used for growing the inoculated seedlings and controls. The trays were arranged in a randomized block design in a controlled environment facility (CEF). The settings for the CEF were as follows: 25/16(±1) ° C. day/night temperature and 65%/85% day/night relative humidity, and a 14-h photoperiod with 500 μmol m-2 s⁻¹ photon flux density at the level of the plant canopy. To promote FCR development, watering was withheld during the FCR assessment. Inoculated seedlings were watered only when wilt symptoms appeared.

Ten trials were carried out to determine FCR response for each of the key recombinant lines identified from the fine-mapping population. Each trial contained two replicates, each with 14 seedlings. Seedlings of 3-day-old were inoculated and FCR severity was assessed with a 0-5 rating at 4 weeks after inoculation, where “0” represents no symptom and “5” whole seedling completely necrotic. The disease ratings from all seedlings for each line in each trial were averaged and used to determine whether the line in concern was resistant or susceptible to FCR infection.

### Linkage Analysis

Linkage analysis was conducted using the software JoinMap® 4.0 (Van Ooijen 2006) with a LOD threshold of 3.0. The Kosambi mapping function was used for converting the recombination frequencies into genetic distances in terms of centimorgan (cM). The genetic linkage map was drawn with the software Mapdraw V2.1.

### Identification of Genes Located in the Targeted Interval

Markers flanking the FCR locus were located on the physical map of the barley pseudomolecule (Mascher et al., 2017) based on the positions of the forward primers (Table 2). Putative genes were arranged and annotated based on information contained in the barley pseudomolecule. Considering the possibility that the gene underlying the resistance at the 4HL locus could be missing in the reference genotype Morex, homologous genes in the corresponding regions of Brachypodium (http://mips.helmholtz-muechen.de/plant/brachypodium/) and rice (http://rice.plantbiology.msu.edu/) were then searched with an e-value cutoff of 10e-10.

### Gene Expression Analysis

RNA-seq data from three pairs of the NILs targeting the Qcrs.cpi-4H locus obtained from an earlier study (Habib et al., 2018) were analysed to analyse the expression of candidate genes located within the targeted region. Based on the fine mapping results described above, CDSs located in the targeted genomic region were retrieved from the barley pseudomolecule. Paired RNA reads from all three sets of the NILs were re-analysed to identify transcripts of interest in the targeted region. RNA datasets were trimmed using SolexaQA scripts (http://solexaqa.sourceforge.net/) to a minimum quality value of 30 and a minimum length of 70.

RNA-seq data from resistant (R line) and susceptible (S line) NILs were analysed against the predicted CDS reference of the barley pseudomolecule (both high and low confidence) using the CLC Genomic Workbench software v9.5 with alignments of >95% coverage and 95% identity. To measure the levels of expression, the quantification of transcript abundance in the samples was calculated by the number of fragments per kilobase of exon per million reads mapped (FPKM) for each of the transcripts (Mortazavi et al., 2008). Single Nucleotide Polymorphisms (SNPs) between the R line, S lines and reference pseudomolecule were investigated. SNPs were identified on the alignment of reads to the reference sequences using the CLC genomic workbench tool “Basic Variant Detection” with >5 coverage and 90% frequency. SNPs between the R and S isolines were identified using the tool of “Compare Sample Variant Tracks”.

### Constructs for Transformation

The full-length CDS for each of the two candidate genes was obtained from the predicted genes model of WBR1. Restriction sites BamHI (GGATCC) and EcoRI (GAATTC) were added to the start and end of each CDS. The CDS of two candidate genes flanked by restriction sites for BamHI (GGATCC) and EcoRI (GAATTC) were synthesized commercially and cloned into the carrier Plasmid pUC57 obtained from GenScript (GenScript USA Inc., Piscataway, NJ, USA). Using the restriction enzymes BamHI and EcoRI, the CDS of the two candidate genes were ligated between the Ubiquitin promoter and tml terminator of vector pWubi-tml vector (Wang and Waterhouse, 2000). For barley transformation, the expression cassette was then transferred into the binary vector pWBVec8 (Wang et al., 1998). Sanger sequencing confirmed the accuracy of the constructs.

### Generation of Transgenic Barley

Agrobacterium transformation of barley was undertaken as described by Tingay et al. (1997) and Jacobsen et al. (2006). Barley cultivar, Golden Promise, plants were propagated [in pots] under glasshouse growth conditions using an 18° C., 16 h light/13° C., 8 h dark growth regime and plants were fertilised with a commercial fertiliser (Osmocote). Barley heads were harvested when developing embryos were 1.5-2 mm in size. Seeds were surface sterilised for 10 min in a 1% sodium hypochlorite solution. Embryos were removed from the seed under aseptic conditions and, after removal of the embryonic axis, scutellum tissue co-cultivated with Agrobacterium strain AGL0 containing a full length CDS encoding a candidate gene in binary vector vec8. Embryos were co-cultivated for 2 days on callus induction medium (Jacobsen et al., 2006) in the dark, without selection. After co-cultivation explants were transferred to callus induction media containing 50 ug/ml of hygromycin and placed in the dark at 24° C. Callus cultures were sub-cultured every two weeks on callus induction media containing 50 μg/ml of hygromycin for 8 weeks. After 8 weeks callus was transferred to FHG media (Jacobsen et al. 2006), containing 30 μg/ml of hygromycin, with a 16-hour 200 μmols m⁻² s⁻¹ light/8-hour dark photoperiod and constant temperature of 24° C. Shoots were transferred to hormone free callus induction media, supplemented with 30 μg/ml of hygromycin, to allow root formation and once a robust root system was developed the T1 plants were then transferred to pots containing potting mix and grown in the glasshouse of CSIRO Canberra site.

Twenty (20) of the T1 barley transgenic plants were progressed to T3 generation by two rounds of self-pollination of the T1 plants. Seeds from individual T3 lines were used for FCR assessment based on the method described above. Each of the T3 lines were assessed in two independent trials. Each trial contained was performed in two replicates, each replicate was with 14 seedlings.

### Example 2—Fine Mapping of the Locus Underlying FCR Resistance

Map-based cloning of the gene underlying FCR resistance at the 4HL locus was based on the two markers flanking the FCR locus identified previously based on the analysis of 1,820 NIL-derived lines (Jiang et al., 2019). The two markers were used to screen the new fine mapping population consisting of 2,203 lines. Key recombinant lines in the targeted region (those with recombination between the two markers) were identified and their levels of FCR resistance assessed using the method described in Example 1.

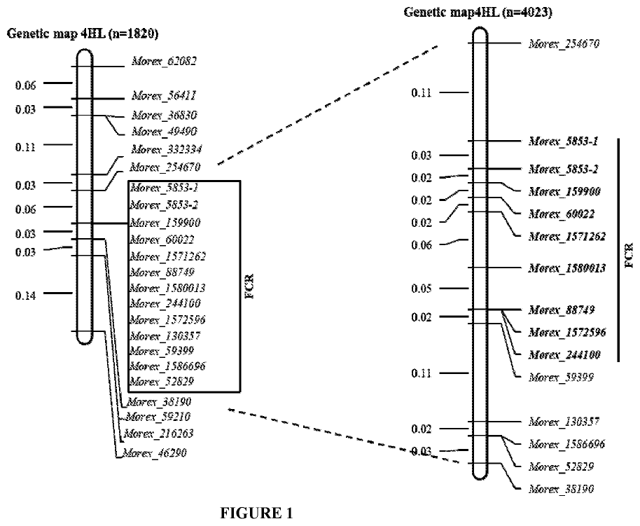

Linkage analysis was conducted based on the phenotypes (FCR resistance) of these recombinant lines and profiles of the markers developed for this interval (Table 2). The results from this linkage analysis are summarised in FIG. 1. As shown, recombination was detected among the 13 co-segregating markers obtained earlier (Jiang et al., 2019) and their order is highly consistent with the physical map of these marker sequences around the 4HL locus. The FCR locus was reliably placed in an interval containing 9 genes (FIG. 1).

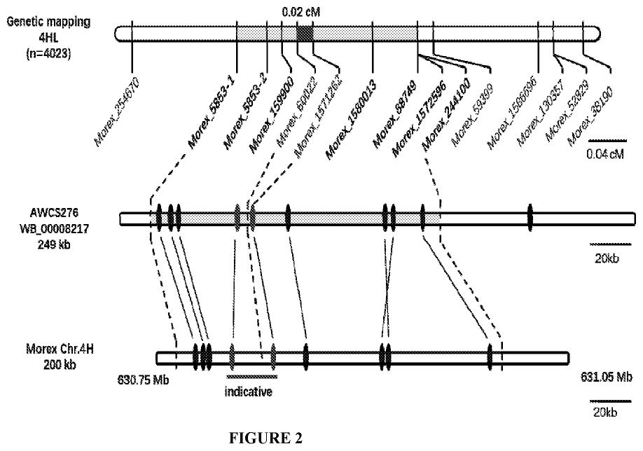

The 9 candidates were located on a single scaffold (249 Kb) in the genome assembly of the R allele donor WBR1. Recombination among the 9 genes was detected. The markers Morex_60022 and Morex_1571262 have a linkage distance of 0.02 cM (FIG. 2). However, both markers co-segregated with the R locus as no recombinant plants with genomic variation were found between the markers. Two candidate genes were suggested from this experiment, the heavy metal transport/detoxification protein superfamily member (WBO1_008217_0052297) and the atypical cinnamyl alcohol dehydrogenase (HvCAD2 WBO1_008217_0065046). The mapping results suggested that either of these two co-segregating genes was responsible for FCR resistance at the Qcrs.cpi-4H locus, thus they were marked as ‘indicative’ (FIG. 2). However, all of the 9 genes remained as candidates due to limited recombination events among them.

### Example 3—Cloning of the Gene Underlying FCR Resistance

### Identification of Candidate Genes

In an effort to identify the gene responsible for FCR resistance at the 4HL locus, the inventors first considered the putative functions of the 9 genes located in the targeted interval. Five encode uncharacterized proteins (Table 3). They were thus not treated as key targets for further assessment.

TABLE 3
Basic information of the 9 genes located in the targeted interval

| | | Fg/mock | Fg/mock | | CDS | |
|---|---|---|---|---|---|---|
| R1 Gene | Morex Gene | (R1) | (S1) | Function | Identities | Note |
| WB01_008217_{—} | HORVU4Hr1G085050 | 0.86 | 0.76 | purple acid | 1848/1854 | |
| 0019035 | | | | phosphatase 27 | | |
| WB01_008217_{—} | HORVU4Hr1G085060 | NA | NA | uncharacterized | Indel | |
| 0025422 | | | | protein | | |
| WB01_008217_{—} | HORVU4Hr1G085070 | 0.73 | 1.03 | uncharacterized | 3352/3375 | |
| 0026673 | | | | protein | | |
| WB01_008217_{—} | HORVU4Hr1G085090 | 1.02 | 1.47 | Heavy metal | 590/591 | No |
| 0052297* | | | | transport/ | | amino |
| | | | | detoxification | | acid |
| | | | | superfamily | | variation |
| | | | | protein | | |
| WB01_008217_{—} | HORVU4Hr1G085100 | 0.93 | 0.68 | Atypical | 1112/1119 | |
| 0065046* | | | | connamoyl-CoA | | |
| | | | | dehydrogenase | | |
| WB01_008217_{—} | HORVU4Hr1G085120 | 0.87 | 0.24 | uncharacterized | 436/438 | |
| 0082413 | | | | protein | | |
| WB01_008217_{—} | HORVU4Hr1G085140 | 11 | 10.72 | remorin family | 1270/1275 | No CDS |
| 0129747 | | | | protein | | variation |
| | | | | | | in NILs |
| WB01_008217_{—} | HORVU4Hr1G085130 | NA | NA | uncharacterized | 257/258 | |
| 0133743 | | | | protein | | |
| WB01_008217_{—} | HORVU4Hr1G085150 | 1 | 0.27 | uncharacterized | 593/597 | |
| 0148062 | | | | protein | | |

The inventors then analysed the expression of the candidate genes using the transcriptome data obtained from three pairs of the NILs in an earlier study (Habib et al., 2018). Expression was not detected from either the inoculated or the non-inoculated controls for two of the uncharacterised genes (Table 3), and the inventors thus concluded these genes were unlikely involved in conferring FCR resistance.

Differences in CDS and predicted amino acid sequences between the R and S isolines of the three NIL pairs were further analysed based on reads mapping. Differences in amino acid sequences were not detected for two of these candidate genes, the Remorin protein and the Heavy metal transport/detoxification superfamily protein (Table 3). It was concluded that these two genes were unlikely involved in conferring FCR resistance.

On the basis of their linkages with the R locus, their putative functions, expression, and differences in CDS and predicted amino acid sequences between R & S isolines of the three NIL pairs, the inventors inferred that the HvCAD2 gene was the most likely candidate underlying FCR resistance at the targeted locus.

### Characterizations of the Candidate Gene and its Structural Analyses

It is known that the CAD catalyses the key reduction reaction in the conversion of cinnamic acid derivatives into monolignol building blocks for lignin polymers in plant cell walls. Alternatively, homologues of the candidate gene encode enzymes catalyses the reduction of flavanones or flavanols. The predominant form of classical CAD belongs to the medium-chain dehydrogenase/reductase (MDR) superfamily and is both NADPH and Zn²⁺ dependent. However, HvCAD2 belong to atypical CAD (Pan et al., 2014), which encodes a predicted protein of 372 amino acids (40.72 kDa) belonging to short-chain dehydrogenase/reductase (SDR) family (cd08958). Wheat, rice, mazie and sorghum all have predicted orthologs of the barley gene (with amino acid identity varies from 66.58% to 81.99%). None of these closest predicted orthologs have been functionally characterized.

Sequence analysis shows that four missense variants were detected at 542, 544, 547 and 551 between R and S alleles in the coding region of HvCAD2, which gave rise to four consecutive amino acids changes in polypeptides. These nucleotide changes results in a change of amino acids from a conserved valine to alanine (position 179, V179A), isoleucine to leucine (position 180, I180L), valine to phenylalanine (position 181, V181F) and asparagine to threonine (position 182, N182T).

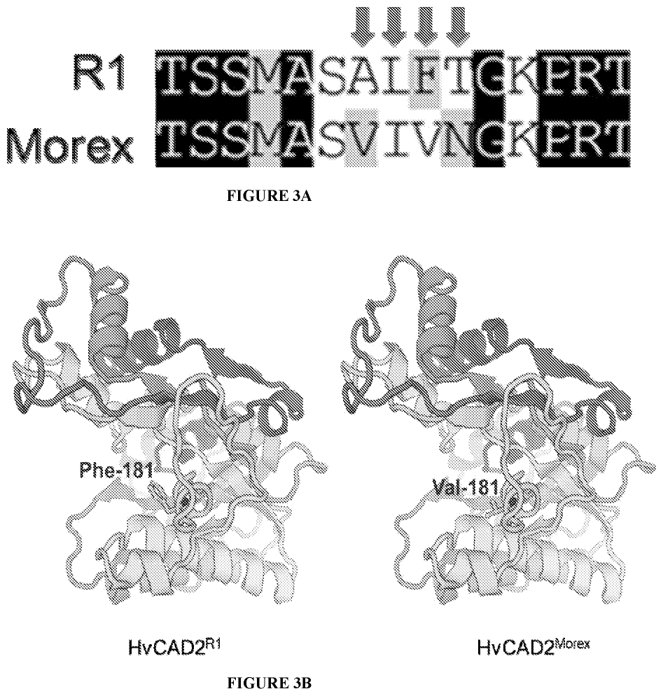

In order to understand the mechanism behind the influence of the observed mutation on protein structure, the inventors analyzed predicted 3D structures of the HvCAD2 protein in the SWISS-MODEL database (FIG. 3B). A homology model of HvCAD2 was generated from the structure of M. truncatula Mt-CAD2 (template 4qtz.1.A), which shares 64.15% sequence identity with HvCAD2. In accordance with Mt-CAD2 binding site for phenylpropene-aldehyde substrate (Pan et al., 2014), position 181 in HvCAD2 were predicted as key substrate binding site. It was demonstrated in the previous study that the catalytic specificity for sinapaldehyde was increased 4-fold in the Mt-CAD2 single-site mutants from tyrosine to phenylalanine at this site (Pan et al., 2014). A similar structural prediction using the Phyre2 server based upon the dihydroflavonal-4-reductase positions these four key amino acids as being either part of the substrate binding site (position 180) or immediately adjacent to the substrate binding site. Thus, four consecutive amino acids changes around the substrate binding site would very likely lead to changes in enzyme activity of HvCAD2.

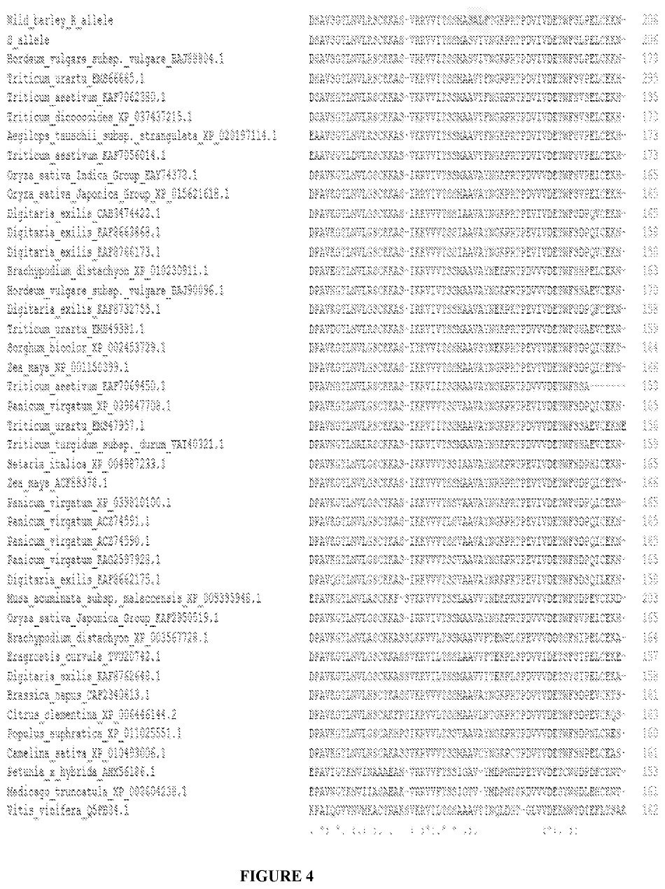

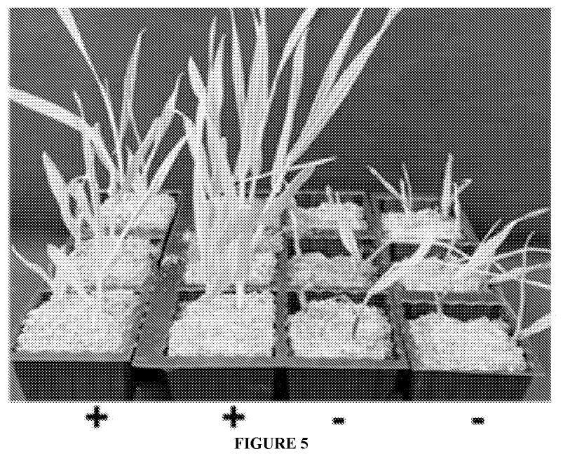

Without exception, homologs of this gene in other plants do not have alanine at the position corresponding to amino acid position 179 of SEQ ID NO: 1 (FIG. 6). The threonine corresponding to amino acid position 182 is also unique to the wild barley resistance allele amongst cereals. Invariably, the homologs had a valine at the position corresponding to amino acid 179 and an asparagine at position 182 (see, for example, the alignments provided in FIGS. 4 and 5). These two amino acids were therefore highly conserved in other CAD2 polypeptides, and the sequence difference in either one, or both, amino acids indicative of an altered function that is the cause of the resistance phenotype to Fusarium pathogens.

### Example 4—Production and Evaluation Of Transgenic Plants Transformation

Based on the method described above, 20 individual T1 plants were obtained. Each of these T1 plants was grown in an individual pot in the PC2 growth rooms in CSIRO Canberra laboratories. Some 100 kernels were harvested from each of the 20 T1 plants (T2 seeds). All the seeds harvested from the T1 transgenic plants were transported to CSIRO St Lucia laboratories for further characterization.

Four T2 seeds from each of the 20 T1 plants were grown in the CEF rooms at CSIRO St Lucia site. They were individually grown in 2.0 litre pots. About 200 T3 seeds were obtained from each of these T2 plants.

### Generation of Transgenic Barley Plants

FCR resistance of a total of 62 T3 lines (sublines) derived from 17 different T1 lines (Table 4), have been assessed. Majority of the lines containing the R allele showed enhanced resistance to FCR (FIG. 5). A small number of the transgenic lines did not show the expected FCR resistance as expected.

### Example 5—Production of Genetically Edited Plants

Comparison of Amino Acid Sequences of the Gene and its Orthologs from Different Species

Following the validation of the gene based on transformation, the inventors analysed orthologs in different plant species including wheat, rice, maize and sorghum. An alignment of related polypeptides from some other plant species is provided in FIG. 6.

Experiments are carried out to modify the genes encoding the endogenous CAD2 to encode mutant polypeptides such that the polypeptide does not have the native amino acid at positions to the Hordeum vulgare susceptible allele sequence. Mutated plants are screened to select for plants with a modification at the amino acid positions corresponding to the valine at position 179, and/or the isoleucine at position 180, and valine at position 181 and/or the asparagine at 182 to convert them into resistant polypeptides. For example the nucleotide changes results in a change of amino acids from a conserved valine to alanine (position 179, V179A), isoleucine to leucine (position 180, I180L), valine to phenylalanine (position 181, V181F) and asparagine to threonine (position 182, N182T). In order to provide resistance genes for these plant species and other plants.

TABLE 4
Genotype and phenotype of transgenic
plants containing the gene CCAR*

| Line | sublines | genotype | phenotype |
|---|---|---|---|
| B64-1 | 1 | a | S |
| | 2 | b | S |
| | 3 | b | S |
| | 4 | a | H/S |
| B64-2 | 1 | a | S |
| | 2 | b | S |
| | 3 | a | R |
| | 4 | a | H/S |
| B64-3 | 1 | b | S |
| | 2 | b | S |
| | 3 | b | S |
| | 4 | b | S |
| B64-4 | 1 | a | R |
| | 2 | a | R |
| | 3 | b | S |
| | 4 | a | R |
| B64-5 | 1 | b | S |
| | 2 | b | S |
| | 3 | b | S |
| | 4 | b | S |
| B64-6 | 1 | a | H? |
| | 2 | a | R |
| | 3 | a | R |
| B64-7 | 1 | b | S |
| | 2 | b | S |
| | 3 | b | S |
| B64-8 | 1 | a | H? |
| | 2 | a | S |
| | 3 | a | R |
| | 4 | a | R |
| B64-9 | 1 | b | S |
| | 2 | a | R |
| | 3 | a | R |
| | 4 | a | H? |
| B64-10 | 1 | b | S |
| | 2 | b | S |
| | 3 | b | S |
| | 4 | b | S |
| B64-11 | 1 | b | S |
| | 2 | b | S |
| | 3 | b | S |
| | 4 | b | S |
| B64-12 | 1 | a | R |
| | 2 | a | R |
| | 3 | a | R |
| | 4 | a | R |
| B64-13 | 1 | a | R |
| | 2 | a | R |
| | 3 | a | H? |
| | 4 | a | H? |
| B64-14 | 1 | a | R |
| | 2 | a | H? |
| | 3 | a | S |
| B64-15 | 2 | b | S |
| | 3 | a | R |
| | 4 | a | R |
| B64-16 | 1 | a | R |
| | 2 | a | R |
| | 3 | a | H? |
| | 4 | a | H? |
| B64-17 | 1 | a | R |
| | 2 | a | H? |
| | 3 | a | R |

*‘a’ indicates plants/lines containing the targeted gene, and ‘b’ those do not contain the gene. Phenotyping and genotyping against the three lines marked in red are being conducted.

### Example 6—Gene Editing of Barley CAD2

A gene editing strategy to create mutations in one or more or all of the endogenous CAD2 to generate mutant polypeptides such that the polypeptide does not have the native amino acid at position 179, 180, 181 and 182 of the Hordeum vulgare susceptible allele sequence could be performed as outlined herein.

### Guide Design

The DNA sequence for the barley crown rot resistance gene was uploaded to Geneious Prime software (version 2021.1.1) and translated to an amino acid sequence. The substrate binding site was annotated in the amino acid sequence (amino acid numbers 173 to 186 of SEQ ID NO:1). CRISPR Cas9 sites using Geneious Prime inbuilt program were identified. Criteria for Cas9 guide targets were analysed (Target site of N(20) and PAM site of NGG) were assessed using the scoring algorithm as described in Doench et al. (2016).

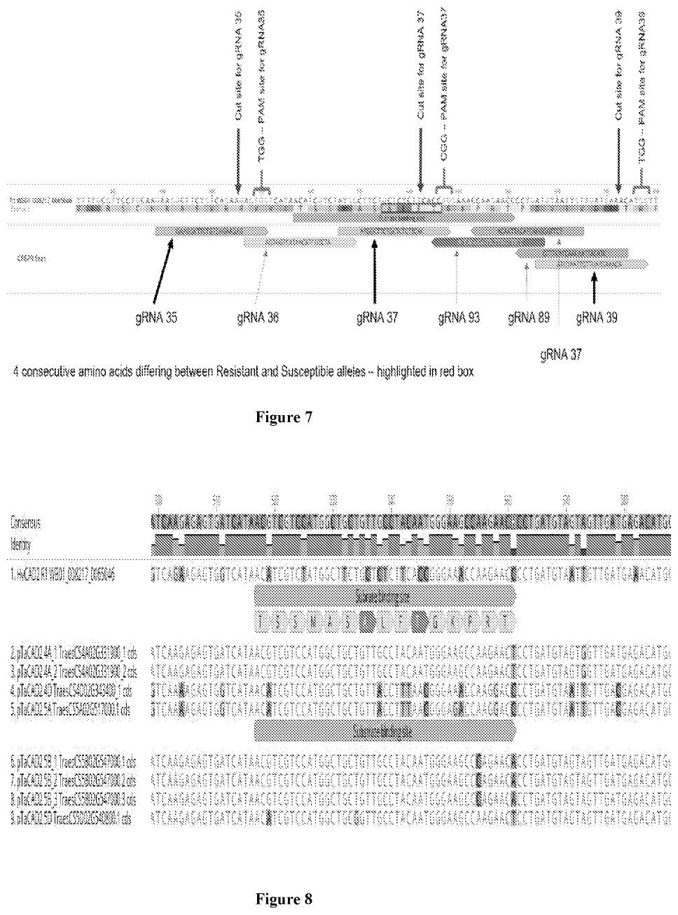

127 gRNA's that are 20 bp in length were identified. A manual inspection of the sequence alignment was then made gRNA's where sequence starts with a ‘T’ or ‘C’ nucleotide which are not compatible with the Polymerase III promoters were discarded. gRNA's starting with an ‘A’ nucleotide are compatible with U3 polymerase III promoters and a ‘G’ nucleotide with U6 polymerase III promoters and were retained. 53 gRNA's were found to fit experimental requirements. gRNA's starting with a ‘T’ or ‘C’ nucleotide are selected then an additional nucleotide needs to be added to the 5′ end, either ‘A’ or ‘G’. To focus on predicted substrate binding site 7 gRNA's were selected (Table 5), as illustrated in FIG. 7.

TABLE 5
Selected gRNA for Barley genomic editing

| | | | | Doench | Doench | Protospacer | |
|---|---|---|---|---|---|---|---|
| | 5′ | 3′ | | (2016) | (2016) | Adjacent | |
| | position | position | Di- | Activity | Python | Motif | |
| Name | of gRNA | of gRNA | rection | Score | Version | (PAM) | gRNA Sequence |
| CRISPR | 489 | 511 | F | 0.644 | 3.8 | TGG | GAAGGCTTCTGTCAG |
| guide | | | | | | | AAGAG (SEQ ID |
| 35 | | | | | | | NO: 52) |
| CRISPR | 507 | 529 | F | 0.469 | 3.8 | TGG | AGTGGTCATAACATC |
| guide | | | | | | | GTCTA (SEQ ID |
| 36 | | | | | | | NO: 53) |
| CRISPR | 526 | 548 | F | 0.434 | 3.8 | CGG | ATGGCTTCTGCTCT |
| guide | | | | | | | CTTCAC (SEQ ID |
| 37 | | | | | | | NO: 54) |
| CRISPR | 566 | 588 | F | 0.589 | 3.8 | TGG | ATGTAATTGTTGAT |
| guide | | | | | | | GAAACA (SEQ ID |
| 39 | | | | | | | NO: 55) |
| CRISPR | 562 | 584 | R | 0.392 | 3.8 | AGG | GTTTCATCAACAA |
| guide | | | | | | | TTACATC (SEQ ID |
| 89 | | | | | | | NO: 56) |
| CRISPR | 545 | 567 | R | 0.314 | 3.8 | CGG | ATCAGGGGTTCTTG |
| guide | | | | | | | GTTTCC (SEQ ID |
| 93 | | | | | | | NO: 57) |

### Constructs for Gene Editing

Single target gRNAs that directly target the four consecutive amino acids differing between Resistant and Susceptible alleles are shown in Table 5. A minimum gene edit at this location of a nucleotide deletion or insertion will cause a frame shift changing the amino acids downstream of this site and consequently protein structure based on the structure analysis herein.

Two targets utilising the highest Doench scoring gRNA's of the 7 gRNA's presented above. Between gRNA 35 and gRNA 39 there are 77 bps between cut sites and is predicted to alter the substrate binding region. Combinations of any of the gRNA's shown in FIG. 7 can be cloned into a single vector.

### Cloning

The gRNA is cloned under the expression of RNA polymerase III promoters selected from pOsU6, pTaU3, pOsU3. Each gRNA is matched to correct RNA polymerase III promoter, as noted in the previous section on the design and selection of gRNA's section. gRNA 37 and gRNA 39 can be used with either pOsU3 or pTaU3. Additional nucleotides may be added to the gRNA sequence for cloning purposes. gRNA oligo pairs are phosphorylated and annealed to each other using a reaction mix of 1 μL each oligo, 1 μL NEB T4 DNA Ligase Buffer (New England Biolabs ‘NEB’, Victoria, Australia), 1 μL 10 mM ATP, 0.5 μL T4 polynucleotide kinase (10 u/μL) (New England Biolabs, Victoria, Australia), 5.5 μL water and incubated at 37° C. for 30 minutes, followed by incubate at 95° C. for 5 min then program thermocycler to decrease temperature by 5° C./min until 25° C. is reached.

The RNA polymerase III promoter vectors are linearised and de-phosphorylate to restrict self-ligation by incubating the following mixture vector [2 μg], NEB 3.1 Buffer [×10]5 μL, 3 μL BsmBI [10 u/μL] diluted in water to 50 μL at 55° C./180 minutes. Followed by addition of 5 μL restriction enzymes mix (1 μL BglII [10 u/μL], 1 μL EcoRI [10 u/μL], 0.5 μL NEB 3.1 2.5 μL Buffer [×10] diluted in water) to the reaction and overnight incubation at 37° C. After the overnight incubation the mixture is de-phosphorylated by the addition of 3 μL rSAP (Shrimp Alkaline Phosphatase (NEB)), incubated at 37° C./30 minutes. The mixture is inactivated by incubating at 65° C./20 minutes. The linearised vector can be extracted following gel separation using a commercially available kit, e.g. QIAEX II gel extraction kit (QIAGEN, Victoria, Australia). The phosphorylated oligos and linearised de-phosphorylated vectors are ligated using 2×blunt/TA ligase master mix (M0367, NEB). 2 μL of the ligation mixture is used to transform chemically competent E. coli cells (NEB 10-β Competent E. coli cells (#C30191). To confirm cloning the protocol from New England Biolabs is followed (as per “Robust Colony PCR from Multiple E. coli strains using One Taq Quick-Load Master Mixes. Yan Xu”).

### Crispr Cas9 Plant Transformation Vectors

Vectors were constructed using Golden Gate protocol 3 of the supplementary information from Cermak et al. (2017). 2 μL of the Golden Gate cloning reaction is transformed into competent E. coli cells. The antibiotic selection is Kanamycin at 30 μg mL⁻¹. The prepared Cas9/RNA polymerase promoters/gRNA vector is transformed via bombardment or Agrobacterium. For barley the transformation follows the published protocol by Tingay et al. (2022). Following plant transformation and the production of transformed plant lines gDNA is assessed for gene editing events. gDNA is extracted from plant tissue with commercially available kits. PCR a region around the potential gene editing sites used herein.

Forward Primer to Barley CAD2 gene at position 255: 5′ GGACACCGCTGACCCAAATA (SEQ ID NO:58)
Reverse Primer to Barley CAD2 gene at position 861: 5′ TGCAAGGATATGTGCCAGGG (SEQ ID NO:59)
PCR product size: 606 bp. PCR products are sequenced with Forward Primer.

### Example 7 Gene Editing of Wheat CAD2

The WBO1_008217_0065046 (HvCAD2^{R1}) sequence was used to blast EnsemblePlants Triticum aestivum (https://plants.ensembl.org/index.html). Within the top 15 results there were matches to the following 5 genes:
1. TraesCS5A02G517000 (pTaCAD2 5A)—94.8% alignment—6 matches
2. TraesCS4D02G343400 (pTaCAD2 4D)—93.4% alignment—6 matches
3. TraesCS5D02G540800 (pTaCAD2 5D)—90.3% alignment—1 match.
4. TraesCS4A02G331900 (pTaCAD2 4A)—90.3% alignment—1 match.
5. TraesCS5B02G547000 (pTaCAD2 5B)—90.3% alignment—1 match

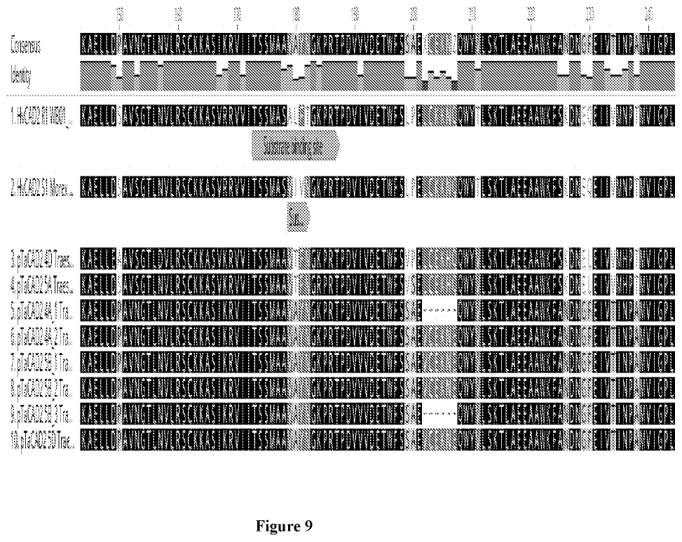

The wheat genome has undergone translocation of sections, hence the HvCAD2^{R1} sequence, has alignments across chromosomes 4 and 5 in wheat. The wheat sequences, genomic, coding domain and amino acid, were downloaded from the EnsemblePlants website. There were 2 coding domain sequence (cds) variants for pTaCAD2 4A and 3 cds variants for pTaCAD2 5B. All coding domain sequences were aligned with HvCAD2R1 and the putative substrate binding site annotated (FIG. 8). There are regions of homology between all sequences and regions with nucleotide differences. The peptide sequence indicates that the amino acids at positions 179 and 182 are the same as that in the susceptible barley amino acid region for all of the wheat sequences (FIG. 9). Two of the variants are missing 6 amino acids downstream of the substrate binding site, but there is no indication if these variants are functional or not.

Design and Selection of gRNA's

To deigns the gRNAs for wheat the WheatCrispr program was used, https://crispr.bioinfo.nrc.ca/WheatCrispr/. This program was used as it can also provide information about off-target locations within the wheat genome. The wheat gene name ‘TraesCS4D02G343400’, was input into the program. The on-target set was to the coding region. All gRNAs that are 20 bp in length were selected, and 125 gRNA's identified. Manual removal of gRNA's where sequence starts with a ‘T’ or ‘C’ nucleotide as indicated in Example 6 these gRNA's are not compatible with our Polymerase III promoters. gRNA's starting with a ‘A’ nucleotide are compatible with U3 polymerase III promoters and ‘G’ nucleotide with U6 polymerase III promoters; 62 gRNA's fit experimental requirements.

Focused assessment of gRNA's around the predicted substrate binding site as follows;
a) 5 gRNA's that will cut within the predicted substrate binding site. gRNA 69, gRNA 119, gRNA 57, gRNA 67, gRNA 95.
b) 6 gRNA's around the predicted substrate binding site. gRNA 88, gRNA 79, gRNA 102, gRNA 44, gRNA 91, gRNA 110 (Table 6).

TABLE 6
Selected gRNA for Wheat genomic editing

| gRNA | Location | Di- | | | overall |
|---|---|---|---|---|---|
| No | of gRNA | rection | gRNA sequence | pam | score |
| 44 | 499452135 | + | AGAGCTATGTGAAAAGCACC | AGG | 0.43 |
| | | | (SEQ ID NO: 60) | | |
| 57 | 499452060 | − | GTTAAAGGTAACAGCAGCCA | TGG | 0.40 |
| | | | (SEQ ID NO: 61) | | |
| 67 | 499452075 | − | GGTCCTTGGTTTCCCGTTAA | AGG | 0.39 |
| | | | (SEQ ID NO: 62) | | |
| 69 | 499452042 | + | AGTGGTCATAACATCGTCCA | TGG | 0.38 |
| | | | (SEQ ID NO: 63) | | |
| 79 | 499452101 | + | ATGTAATTGTTGACGAGACA | TGG | 0.37 |
| | | | (SEQ ID NO: 64) | | |
| 88 | 499452024 | + | GAAAGCTTCTGTCAAAAGAG | TGG | 0.36 |
| | | | (SEQ ID NO: 65) | | |
| 91 | 499453327 | + | GTCCAAGACCCTTGCAGAGG | AGG | 0.35 |
| | | | (SEQ ID NO: 66) | | |
| 95 | 499452089 | − | ACAATTACATCAGGGGTCCT | TGG | 0.35 |
| | | | (SEQ ID NO: 67) | | |
| 102 | 499452098 | − | GTCTCGTCAACAATTACATC | AGG | 0.34 |
| | | | (SEQ ID NO: 68) | | |
| 110 | 499453330 | − | AGCCTCCTCTGCAAGGGTCT | TGG | 0.32 |
| | | | (SEQ ID NO: 69) | | |
| 119 | 499452061 | + | ATGGCTGCTGTTACCTTTAA | CGG | 0.30 |
| | | | (SEQ ID NO: 70) | | |

To further aid selection of the best fit guides each proposed gRNA sequence was aligned to each gene homologue, identified herein. The potential for the Cas9 enzyme to cut at that position on the genome and the number of nucleotide mismatches with each homologue was assessed (Table 7). Mismatches of the gRNA to the genome may or may not result in a gene edit at that location. For example, gRNA 57. The pTaCAD2 4D and pTaCAD2 5A sequence matches the pTaCAD2 4D sequence and all three genes could have gene edits with the CRISPR technology. The pTaCAD2 4A, pTaCAD2 5B and pTaCAD2 5D sequences have nucleotide differences with the pTaCAD24D sequence, gene edits on these genes is less certain.

TABLE 7
Summary of results assessing the potential gRNA's around
the putative substrate binding site and information on
potential CRISPR gene editing across all gene copies.

| | | |
|---|---|---|
| Consider | gRNA 57 | 4/5 mismatches in 4A, 5B, 5D |
| | gRNA 69 | 1/2 mismatches in 4A, 5B, 5D |
| | gRNA 95 | 2/3 mismatches in 4A, 5B, 5D |
| | gRNA 79 | 3 mismatches in 4A, 5B, 5D |
| | gRNA 102 | 3 mismatches in 4A, 5B, 5D |
| | gRNA 110 | 2 mismatches in 4A, 5B, 5D |
| discard | gRNA 67 | Largest # mismatches (6) including 5A (1) |
| | gRNA 119 | Mismatch in critical zone positions 3-4 |
| | gRNA 88 | No PAM site in 4A, 5B, 5D |
| | gRNA 44 | Unsure if PAM site exists |
| | gRNA 91 | No PAM site in 4A, 5B, 5D |

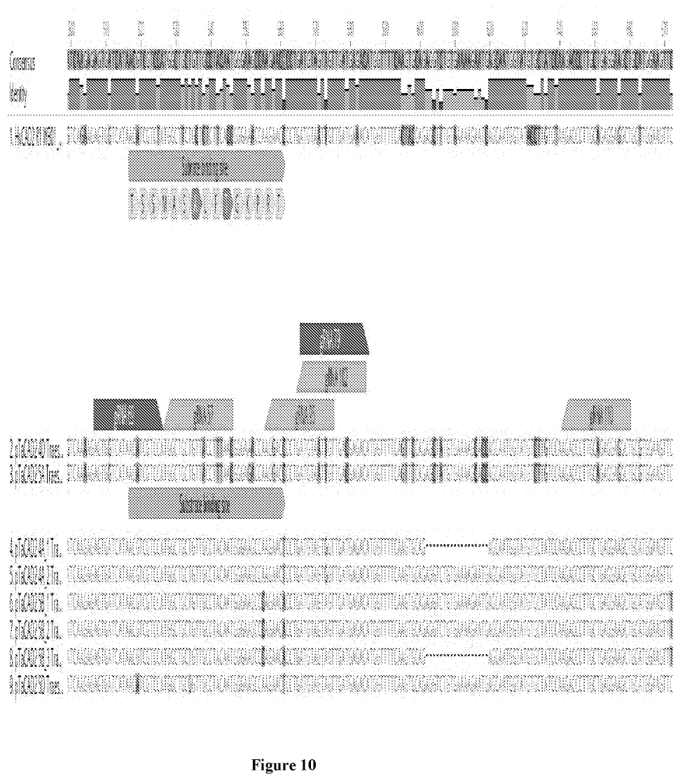

The alignment of the gRNA's as potential targets across all gene copies are aligned in FIG. 10. The following constructs were proposed for gene editing at the substrate binding site:

$$a . U 3 - gRNA 69 + U 6 - gRNA 57 + U 3 - gRNA 95 + U 3 - gRNA 79$$

$$b . U 3 - gRNA 69 + U 6 - gRNA 57 + U 3 - gRNA 95 + U 6 - gRNA 102$$

gRNA 57 provides a single gRNA directly targeting the 4 consecutive amino acids differing between Resistant and Susceptible alleles. Combinations of any of the gRNA's highlighted in the Figure can be cloned into a single vector.

The Cas9/RNA polymerase promoters/gRNA vectors for plant transformation were prepared as described in Example 6 adapted as needed to use the wheat sequences. Example primer combinations for proposed construct OsU3-gRNA 69+OsU6-gRNA 57+TaU3-gRNA 95+OsU6-gRNA 102. PCR reaction 1 use primers OsU3WCRg69top and TaU3WCRg95bot. PCR reaction 2 use primers OsU3WCRg57top and OsU6WCRg102bot (Table 8).

TABLE 8
Primers

| | | | Wheat |
|---|---|---|---|
| Vector | Oligo name | Sequence | gRNA |
| pOsU3 | OsU3WCRg69top | 5′ TGGCAGTGGTCATAACATCGTCCA (SEQ ID | gRNA 69 |
| | | NO: 71) | |
| | | 3′ TCACCAGTATTGTAGCAGGTCAAA | |
| | | 5′ (reverse of (SEQ ID NO: 72) | |
| | OsU3WCRg69bot | 5′ AAACTGGACGATGTTATGACCACT 3′ (SEQ | |
| | | ID NO: 72) | |
| pOsU6 | OsU6WCRg57top | 5′ GTGTTGGCTGCTGTTACCTTTAAC (SEQ ID | gRNA 57 |
| | | NO: 73) 3′ ACCGACGACAATGGAAATTGCAAA 5′ | |
| | | (reverse of (SEQ ID NO: 74) | |
| | OsU6WCRg57bot | 5′ AAACGTTAAAGGTAACAGCAGCCA 3′ (SEQ | |
| | | ID NO: 74) | |
| pTaU3 | TaU3WCRg95top | 5′ TAGCAGGACCCCTGATGTAATTGT (SEQ ID | gRNA 95 |
| | | NO: 75) 3′ TCCTGGGGACTACATTAACACAAA 5′ | |
| | | (reverse of (SEQ ID NO: 76) | |
| | TaU3WCRg95bot | 5′ AAAC ACAATTACATCAGGGGTCCT (SEQ ID | |
| | | NO: 76) | |

### Wheat Transformation

Grow wheat cultivars in a glasshouse using 24° C. 16 h light/18C, 8 h dark growth cycle. Plants are grown in potting mixture and fertilised fortnightly with Aquasol. Wheat heads are tagged at anthesis and harvested 12-14 days post anthesis for transformation experiments.

Agrobacterium strains and triparental mating follow protocols described in Richardson et al. (2014). Wheat transformation using Agrobacterium tumefaciens is undertaken as described by Ishida et al. (2015) as modified by Richardson et al. (2014). Briefly, seeds are harvested 12-14 days post-anthesis then surface sterilised in a 0.8% sodium hypochlorite solution for 10 min. Embryos are removed from the seed under aseptic conditions and co-cultivated with Agrobacterium strains containing binary constructs of interest for 2 days on WLS-AS medium (Ishida et al., 2015) in the dark. After co-cultivation embryonic axes are excised with a scalpel and explants are then transferred to WLS-Res medium and placed in the dark at 24° C. After 5 days transfer explants to WLS-P5 callus induction media containing 5 mg/ml of phosphinothricin (PPT). Two weeks later callus is bisected and placed on WLS-P10 (10 mg/l of PPT) for 3 weeks in the dark. Callus is then regenerated on LSZP5 (5 mg/l PPT) medium in 200 umols m-2 s⁻¹ light at 24° C. Transfer shoots to LSF-P5 (5 mg/l PPT) medium to allow root formation and once robust root systems develop transfer plants to the glasshouse.

### Confirmation of Genome Editing

Following plant transformation and the production of transformed plant lines gDNA is assessed by extracting gDNA from leaf tissue of recovered plants, followed by PCR of the region around the gRNA and sequencing the PCR product. There are several programs that can assist with identifying gene edits and designing PCR primers. Suggested primers to amplify around the putative substrate binding region as has been the focus of examples within this document. Forward primer to pTaCAD2 4D gene at position 208: AACGATAGGCTGCAGCTGTT (SEQ ID NO:77). Reverse primer to pTaCAD2 4D gene at position 891: CTCGTCATCTCCACGCTTGT (SEQ ID NO:78). Expected PCR product size 683 bp.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU 2021902650 filed 23 Aug. 2022, the entire contents of which are incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4:1087.
Bam et al. (2008) Proc S Afr Sug Technol Ass 81:508-512.
Barker et al. (1983) Plant Mol. Biol. 2: 235-350.
Begemann et al. (2017) Sci Rep. 7(1):11606.
Bevan et al. (1983) Nucl. Acid Res. 11: 369-385.
Cadwell and Joyce (1992) PCR Methods Appl. 2:28-33.
Capecchi (1980) Cell 22:479-488.
Cermak et al. (2017) The Plant Cell 6:1196-1217.
Chakraborty et al. (2006) Australas Plant Path 35:643-655
Chen et al. (2013) PLoS ONE 8(3): e58040.
Cheng et al. (1996) Plant Cell Rep. 15:653-657.
Clapp (1993) Clin. Perinatol. 20:155-168.
Coco et al. (2001) Nature Biotechnology 19:354-359.
Coco et al. (2002) Nature Biotechnology 20:1246-1250.
Comai et al. (2004) Plant J 37: 778-786.
Crameri et al. (1998) Nature 391:288-291.
Curiel et al. (1992) Hum. Gen. Ther. 3:147-154.
Doench et al. (2016) Nature Biotechnology 34:184-191.
Doudna and Charpentier (2014) Science 28:346(6213):1258096.
Eggert et al. (2005) Chembiochem 6:1062-1067.
Eglitis et al. (1988) Biotechniques 6:608-614.
Fujimura et al. (1985) Plant Tissue Cultural Letters 2:74.
Garfinkel et al. (1983) Cell 27: 143-153.
Graham et al. (1973) Virology 54:536-539.
Grant et al. (1995) Plant Cell Rep. 15:254-258.
Greve (1983) J. Mol. Appl. Genet. 1:499-511.
Habib et al. (2016) Euphytica 209:555-563.
Habib et al. (2018) Theoretical & Applied Genetics 131:613-624.
Haft et al. (2005) Computational Biology, PLoS Comput Biol 1(6):e60.
Harayama (1998) Trends Biotechnol. 16:76-82.
Hellinga (1997) Proc. Natl. Acad. Sci. 94:10015-10017.
Henikoff et al. (2004) Plant Physiol 135: 630-636.
Hinchee et al. (1988) Biotech. 6:915.
Ishida et al. (2015) Methods Mol Biol. 1223:189-198.
Jacobsen et al. (2006) Methods in Molecular Biology 343: 171-183.
Jézéquek et al. (2008) Biotechniques 45:523-532.
Jiang et al. (2019) Theoretical & Applied Genetics 132:217-225.
Jinek et al. (2012) Science 337:816-821.
Joshi (1987) Nucl. Acid Res. 15: 6643-6653.
Kazan and Gardiner (2018). Mol Plant Pathol. 19:1547-62.
Langridge et al. (2001) Aust. J. Agric. Res. 52: 1043-1077.
Lemieux (2000) Current Genomics 1: 301-311.
Leung et al. (1989) Technique 1:11-15.
Li et al. (2008) Journal of Phytopathology 156:751-754.
Liang et al. (2017) Nat Commun. 8:14261.
Liang et al. (2018) Plant Biotechnol J. 16:2053-2062.
Liang et al. (2019) Methods Mol Biol. 1917:327-335.
Liu and Ogbonnaya (2015) Plant Breeding 134:365-372.
Lu et al. (1993) J. Exp. Med. 178: 2089-2096.
Luo et al. (2016) Plant Cell Rep 35(7):1439-1450.
Ma et al. (2015) Molecular Plant 8: 1274-1284.
Makarova (2015) Nat. Rev. Microbiol. 13:722-736.
Mascher et al. (2017) Nature 544:427-433
Medberry et al. (1992) Plant Cell 4: 185-192.
Medberry et al. (1993) Plant J. 3: 619-626.
Mitter et al. (2006) Plant Pathol 55:433-441.
Mortazavi et al. (2008) Nat Methods 5:621-628.
Moummou et al. (2012) BMC Plant Biology 12:219.
Needleman and Wunsch (1970) J. Mol Biol. 45:443-453.
Ness et al. (2002) Nature Biotechnology 20:1251-1255.
Ostermeier et al. (1999) Nature Biotechnology 17:1205-1209.
Ow et al. (1986) Science 234: 856-859.
Pan et al. (2014) Plant Cell 26: 3709-3727.
Prasher et al. (1985) Biochem. Biophys. Res. Comm. 126: 1259-68.
Richardson et al. (2014) Plant Cell Tiss Organ Cult 119: 647-659.
Sieber et al. (2001) Nature Biotechnology 19:456-460.
Slade and Knauf (2005) Transgenic Res. 14: 109-115.
Smiley et al. (2005) Plant Dis. 89:595-604.
Stalker et al. (1988) Science 242:419-423.
Stemmer (1994a) Proc. Natl. Acad. Sci. USA 91:10747-10751.
Stemmer (1994b) Nature 370(6488):389-391.
Sun et al. (2016) Molecular Plant 9: 628-631.
Svitashev et al. (2016) Nat Commun. 7:13274.
Thillet et al. (1988) J. Biol. Chem. 263:12500.
Tingay et al. (1997) Plant J 11:1369-1376.
Toriyama et al. (1986) Theor. Appl. Genet. 205:34.
Tunali et al. (2008) Plant Disease 92:1299-1306.
Van Ooijen J W (2006) JoinMap® 4, software for the calculation of genetic linkage maps in experimental populations. Kyazma B V, Wageningen.
Volkov et al. (1999) Nucleic Acids Research 27:e18.
Wagner et al. (1992) Proc. Natl. Acad. Sci. USA 89:6099-6103.
Wang et al. (1998) Tropical Subtropical Species—Part II, 461:401-407.
Wang and Waterhouse (2000) Plant Mol. Biol. 43:67-82.
Woo et al. (2015) Nat Biotechnol. 33:1162-1164.
Zhao et al. (1998) Nature Biotechnology 16:258-261.
Zheng et al. (2021) (submitted to Euphytica)

1-65. (canceled)

66. A plant having a genetically modified gene encoding an atypical cinnamoyl-CoA dehydrogenase 2 (CAD2) polypeptide, wherein when expressed in the plant the polypeptide confers enhanced resistance to one or more biotrophic fungal pathogen(s) when compared to a corresponding plant lacking the gene.

67. The plant of claim 66, wherein the polypeptide comprises amino acids having a sequence at least 90% identical to the amino acid sequence of any one or more of SEQ ID NO's 1 to 10.

68. The plant of claim 66, wherein the genetically modified gene is an exogenous polynucleotide encoding the polypeptide.

69. The plant of claim 66, wherein the one or more fungal pathogen(s) is a rot, rust or a mildew.

70. The plant of claim 66 which is a cereal plant or a legume plant.

71. A process for identifying a polynucleotide encoding a polypeptide which confers enhanced resistance to one or more fungal pathogen(s) to a plant, the process comprising:
i) obtaining a polynucleotide of claim 72 operably linked to a promoter,
ii) introducing the polynucleotide into a plant,
iii) determining whether the level of resistance to one or more fungal pathogen(s) is increased relative to a corresponding plant lacking the polynucleotide, and
iv) optionally, selecting a polynucleotide which when expressed produces a polypeptide which confers enhanced resistance to one or more fungal pathogen(s).

72. An isolated and/or exogenous polynucleotide encoding a polypeptide which confers enhanced resistance to one or more fungal pathogen(s), wherein the polypeptide comprises amino acids having a sequence at least 40% identical to the amino acid sequence of any one or more of SEQ ID NO's 1 to 10 and 79 to 83.

73. A chimeric vector comprising the polynucleotide of claim 72, wherein the polynucleotide is operably linked to a promoter.

74. A recombinant cell comprising an exogenous polynucleotide of claim 72.

75. The cell of claim 74 which is a cereal plant cell or a legume plant cell.

76. A method of producing a polypeptide, the method comprising expressing in a cell or cell free expression system the polynucleotide of claim 72.

77. A method of producing a plant with a genetic modification(s) of claim 66, the method comprising the steps of
i) introducing a genetic modification(s) to a plant cell such that the cell is capable of producing an atypical cinnamoyl-CoA dehydrogenase 2 (CAD2) polypeptide that confers upon the plant comprising the cell enhanced resistance to one or more biotrophic fungal pathogen(s) when compared to a corresponding plant lacking the genetic modification(s),
ii) regenerating a plant with the genetic modification(s) from the cell, and
iii) optionally harvesting seed from the plant, and/or
iv) optionally producing one or more progeny plants from the genetically modified plants, thereby producing the plant.

78. A method of producing a plant with a genetic modification(s) of claim 66, the method comprising the steps of
i) crossing two parental plants, wherein at least one plant comprises a genetic modification(s) of claim 66,
ii) screening one or more progeny plants from the cross in i) for the presence or absence of the genetic modification(s), and
iii) selecting a progeny plant which comprise the genetic modification(s), thereby producing the plant.

79. A method for identifying a plant which has enhanced resistance to one or more fungal pathogen(s), the method comprising the steps of
i) obtaining a sample from a plant, and
ii) screening the sample for the presence or absence of an atypical cinnamoyl-CoA dehydrogenase 2 (CAD2) polypeptide which when expressed in the plant the polypeptide confers enhanced resistance to one or more biotrophic fungal pathogen(s) when compared to a corresponding plant lacking the gene, and/or screening the sample for the presence or absence of the polypeptide.

80. A seed of the plant of claim 66, wherein the seed comprises the genetic modification(s).

81. A method of producing a plant part, the method comprising,
a) growing a plant of claim 66, and
b) harvesting the plant part.

82. A method of producing flour, wholemeal, starch or other product obtained from seed, the method comprising;
a) obtaining seed of claim 80, and
b) extracting the flour, wholemeal, starch or other product.

83. A product produced from a plant of claim 66 and/or a seed therefrom, wherein the product is a food product or beverage product.

84. A method of preparing a product of claim 83, the method comprising mixing seed, or flour, wholemeal or starch from the seed, with another food ingredient.

85. A method of preparing malt, comprising the step of germinating seed of claim 80.


SEQUENCE LISTING (88 sequences)

SEQ ID NO: 1 - protein, 372 aa, Hordeum vulgare
MAPASHQHKPALLDRGGDAETRAERASSHQKKAMAEKQRQGLNVEEGSSGAAGKLVCVTGASGYIASWIVKLLLDRGYTVHGTVRDTADPNKTLHLRALDGANDRLHLFNANLLEEGSFDAAIDGCECVFHAASPVFFAAKDPKAELLDSAVSGTLNVLRSCKKASVRRVVITSSMASALFTGKPRTPDVIVDETWFSLPELCKKNQQWYTLSKTLAEEAAWKFSKDNEQEIIVMNPTMVIGPLLQPTLNASVEAVLNLINGSSPTCPNYAHGWVNVRDVALAHILAYEVPSANGRYCIVERVAHYSELVKIICKMYPNIPLPDKCADDEPLFPTYQVSKDKIRSLGMELIPLETSIKETIESFKEKGFVAF

SEQ ID NO: 2 - protein, 372 aa, Hordeum vulgare
MAPASHQHKPALLDRGGDAETRTERASSHQKKAMAEKQRQGLNVEEGSSGAAGKLVCVTGASGYIASWIVKLLLDRGYTVHGTVRDTADPNKTLHLRALDGANDRLHLFNANLLEEGSFDAAIDGCECVFHAASPVFFAAKDPKAELLDSAVSGTLNVLRSCKKASVRRVVITSSMASVIVNGKPRTPDVIVDETWFSLPELCKKNQQWYTLSKTLAEEAAWKFSKDNEQEIIVMNPTMVIGPLLQPTLNASVEAVLNLINGSSPTCPNYAHGWVNVRDVALAHILAYEVPSANGRYCIVERVAHYSELVKIICKMYPNIPLPDKCADDEPLFPTYQVSKDKIRSLGMELIPLETSIKETIESFKEKGFVAF

SEQ ID NO: 3 - protein, 361 aa, Triticum aestivum
MALAWHQQRPPPGGRYSQRDLAMAEKQQQGSNVEEGSSTAAGKVVCVTGASGYIASWIVKLLLDRGYTVHGTVRDTADPNKTLHLRALDGANDRLQLFNANLLEEGSFDAAIDGCECVFHAASPVFFTIKDPKAELLDSAVSGTLNVLRSCKKASVKRVVITSSMAAVTFNGRPRTPDVIVDETWFSVSELCEKHQQWYVLSKTLAEEAAWKFSKDNELEIVVMHPTMVIGPLLQPTLNASVEVILNLINGSSSTYPNFAHGWVNVKDVALAHILAYEVPSADGRYCIVERVVHYLELVKIISKMYPNISLPYKCGDGEPLFPTYQVWTDKIRSLGVELIPLEKTIKETIESLKEKGFFAL

SEQ ID NO: 4 - protein, 339 aa, Triticum aestivum
MAEKQQQGSNVEEGSSRAAGKVVCVTGASGYIASWIVKLLLDRGYTVHGTVRDTADPDKTWHLRALDGANDRLQLFNANLLEEGSFDAAIDGCECVFHAASPVFFTIKDPKAELLEAAVSGTLDVLRSCKKASVKRVVITSSMAAVTFNGKPRTPDVIVDETWFSVPELCEKHQQWYVLSKTLAEEAAWKFSKDNELEIVVMHPTMVIGPLLQPTLNASVEVILNLINGSSSTYPNIAHGWVNVKDVALAHILAYEVPSADGRYCIVERVAHYLELVKIISKMYPNIPLPYKRGDDEPLFPTYQVSTDKIRSLGVELIPLETTIKETIESLKERGFFEF

SEQ ID NO: 5 - protein, 324 aa, Triticum aestivum
MSSGMGKVVCVTGGSGYIASWLVKLLLHRGYTVRATVRDTADPKKTLHLQALDGAKDRLHLFKASLLEEGSFDAAIAGSDCVFHTASPFYHNVKDPKAELLDPAVNGTLNVLRSCKKASIKRVIITSSMAAVAYNGKPRTPDVVVDETWFSSAEQWYVLSKTLAEEAAWKFANDNGFEIVTINPAMVIGPLLQPTLNTSAEAILKFINGSSSTYPNFSFGWINVKDVALAHILAYEVPSANGRYCMVERVAHHSEIVKIIHEMYPNFPVPEKCADDAPFVPIYQVSKDKIRSLGMELIPLETSLKETIESLREKGFVTSESSHL

SEQ ID NO: 6 - protein, 336 aa, Oryza sativa
MSSESEAAPGTGKLVCVTGASGYIASWLVRLLLARGYTVRATIRDTSDPKKTLHLRALDGANERLHLFEANLLEEGSFDAAVNGCDCVFHTASPFYHNVKDPKAELLDPAVKGTLNVLGSCKKASIRRVIVTSSMAAVAYNGKPRTPDVVVDETWFSVPEICEKHQQWYVLSKTLAEEAAWKFSKDNGFEIVTVNPAMVIGPLLQPSLNTSAEAILKLINGSSSTYPNFSFGWINVKDVALAHILAYEVPSANGRYCMVERVAHYSELVQIIREMYPNIPLPDKCADDKPSVPIYQVSKEKIKSLGLELTPLHTSIKETIESLKEKGFVTFDSSNL

SEQ ID NO: 7 - protein, 332 aa, Zea mays
MASAAATATMGTGKVVCVTGASGYIASWIVRLLLDRGYTVRATVRDTADPKKTLHLTALDGAKDRLHLFKASLLEEGSFDAAVHGCDTVFHTASPFYHNVKDAKAELLDPAVKGTLNVLGSCKKASIKKVVVTSSMAAVAYNGRPRTPEVTVDETWFSDPQICETNQQWYILSKTLAEEAAWKFSRDNGLEIVTINPAMVIGPLLQPTLNTSAEAILKLINGSSSTYPNFCFGWVNVKDVALAHILAYEVPSSNGRYCMVERVVHYSELVNIIRNMYPTLPLPDKCADDKPFVPPYQVSKEKIKSIGIELIPLETSVKETIESLKEKGFASF

SEQ ID NO: 8 - protein, 334 aa, Sorghum bicolor
MSSAAAMTGAGKVVCVTGASGYIASWIVRLLLDRGYTVRATVRDTADPKKTLHLTALDGAKDRLHLFKASLLEEGSFDAAVHGCDTVFHTASPFYHNVKDPKAELLDPAVKGTLNVLGSCKKASITKVVITSSMAAVSYNEKPRTPEVTVDETWFTDPQICEKTQQWYVLSKTLAEQAAWKFSRDNGFEIVTINPAMVIGPLLQPTLNTSAEAILKLINGSSTYPNSSFGWVNVKDVALAHILAYEIPSANGRYCMVERVVHYSELVKIIRKMYPTIRLPDKCADDKPFVPTYQVSKEKIRSIGIELIPVETSVKETIESLKEKGFVSFDSSNL

SEQ ID NO: 9 - protein, 326 aa, Medicago truncatula
MSSSNLGNVVCVTGASGYIASWLVRLLLHRGYTVKATVRDPNDPKKVDHLVKLDGAKERLQLFKANLLEEGAFDSVVQGCHGVFHTASPFYHDVKDPRAELIDPALKGTLNVLNSCAKSSSLKRVVLTSSIAAVAYNGKPRTPDVVVDETWFTDADFCAKSNLWYVVSKTLAEEAAWKFVKENNIDMVTINPAMVIGPLLQPVLNTSAAAILNLINGAQTFPNASFGWVNVKDVANAHILAYENASASGRHCLVERVAHYSEVVRILRELYPSLQLPEKCADDKPYVPIYQVSKEKAKSLGLEYTPLEVSIKETVESLKEKKFANL

SEQ ID NO: 10 - protein, 330 aa, Brassica napus
MANSGEGKVVCVTGASGYIASWLVKFLLSRGYTVKASVRDPSDPKKTQHLVSLDGAKERLHLFKADLLEEGSFDSAIDGCEGVFHTASPFYHDVKDPQAELIDPAVKGTLNVLNSCTKASSVKRVVVTSSMAAVAYNGKPRTPDVTVDETWFSDPEVCKTSKMWYVLSKTLAEDAAWKFAKEKGLDIVTINPAMVIGPLLQPTLNTSAAAILNFINGAKTFSNSTFGWVNVKDVANAHIQAYEIPSANGRYCMVERVLHHSEIVNILRQLYPNLQLPESCADENPFVPTYEVSKEKIKSIGIDYIPLEVSIKETVESLKEKGFINVRASI

SEQ ID NO: 11 - DNA, 1119 bp, Hordeum vulgare
  source 1..1119: mol_type = other DNA
atggcgcctgcttcgcaccaacataaacccgccctcctcgaccgcgggggagatgcagagacaagggctgaacgcgcctcatcgcaccaaaagaaagccatggcagagaagcagcgacaaggactgaacgtggaggaggggagctccggagcagcggggaagctcgtctgcgtcaccggcgcgtccgggtacatcgcgtcctggatcgtcaagctcctcctcgaccgcggctacaccgtccatggcaccgtccgggacaccgctgacccaaataaaacattgcacctgcgtgctcttgatggagcaaacgataggctgcacctgttcaacgcaaacttgttagaggaaggctctttcgacgctgccattgacggttgcgagtgtgtgttccatgcggcttctccagttttcttcgctgccaaagatcccaaggctgagttacttgattcagcagtcagtggaacgcttaatgttttgcgttcctgcaagaaggcttctgtcagaagagtggtcataacatcgtctatggcttctgctctcttcaccgggaaaccaagaacccctgatgtaattgttgatgaaacatggttttcactaccagagctttgtaaaaagaatcagcaatggtataccttgtctaagacccttgcagaggaggctgcttggaagttctctaaagataatgaacaagaaataattgtaatgaaccccacaatggtcattggtcctctactgcagcctacacttaacgctagtgttgaagcagtcttaaacctaatcaatggatcatctcctacatgccctaattatgcccatggctgggtaaatgtcagagatgtggccctggcacatatccttgcatatgaagttccttcagcaaatggaagatattgcattgttgaaagagttgctcattactcggagcttgtcaagatcatatgcaagatgtatccgaacattccactcccagataagtgtgcagatgatgaaccactttttccaacctaccaagtatccaaggacaagataagaagcttgggtatggaactgattcctctagagactagcattaaagagactatagaaagcttcaaagaaaagggttttgttgcattctga

SEQ ID NO: 12 - DNA, 1119 bp, Hordeum vulgare
  source 1..1119: mol_type = other DNA
atggcgcctgcttcgcaccaacataaacccgccctcctcgaccgcgggggagatgcagagacaaggactgaacgcgcctcatcgcaccaaaagaaagccatggcagagaagcagcgacaaggactgaacgtggaggaggggagctccggagcagcggggaagctcgtctgcgtcaccggcgcgtccgggtacatcgcgtcctggatcgtcaagctcctcctcgaccgcggctacaccgtccatggcaccgtccgggacaccgctgacccaaataaaacattgcacctgcgtgctcttgatggagcaaacgataggctgcacctgttcaacgcaaacttgttagaggaaggctctttcgacgctgccattgacggttgcgagtgtgtgttccatgcggcttctccagttttcttcgctgccaaagatcccaaggctgagttacttgattcagcagtcagtggaacgcttaatgttttgcgttcctgcaagaaggcttctgtcagaagagtggtcataacatcgtctatggcttctgttatcgtcaacgggaaaccaagaacccctgatgtaattgttgatgaaacatggttttcactaccagagctttgtaaaaagaatcagcaatggtataccttgtctaagacccttgcagaggaggctgcttggaagttctctaaagataacgaacaagaaataattgtaatgaaccccacaatggtcattggtcctctgctgcagcctacacttaacgctagtgttgaagcagtcttaaacctaatcaatggatcatctcctacatgccctaattatgcccatggctgggtaaatgtcagagatgtggccctggcacatatccttgcatatgaagttccttcagcaaatggaagatattgcattgttgaaagagttgctcactactcggagcttgtcaagatcatatgcaagatgtatccgaacattccactcccagataagtgtgcagatgatgaaccactttttccaacctaccaagtatccaaggacaagataagaagcttgggtatggaactgattcctctagagactagcattaaagagactatagaaagcttcaaagaaaagggttttgttgcattctga

SEQ ID NO: 13 - DNA, 1086 bp, Triticum aestivum
  source 1..1086: mol_type = other DNA
atggcgcttgcttggcaccaacaaagacctcctcctggaggtaggtactcccaaagagacttggccatggcagagaagcagcaacaaggatcgaacgtggaggaggggagctccacggctgcggggaaggtcgtctgcgtcaccggcgcctctgggtatatcgcgtcctggatcgtcaagctccttctcgatcgcggctacaccgtccacggcaccgtccgggacaccgctgacccaaataaaacattgcacctgcgtgctcttgatggagcaaacgataggctgcagctgttcaatgcaaacttgttagaagaaggctctttcgacgctgccattgacggttgcgagtgcgtcttccatgcggcttctccagttttcttcactatcaaggatcccaaggctgagttacttgattcagcggtcagtgggacacttaacgtgctgcgttcctgcaagaaagcttctgtcaaaagagtggtcataacatcgtccatggctgctgttacctttaacgggagaccaaggacccctgatgtaattgttgacgagacatggttttcagtttcagagctatgtgaaaagcaccagcaatggtatgtcttgtccaagacccttgcagaggaggctgcgtggaagttctctaaagataatgaacttgaaatagttgtaatgcacccaacaatggtcatcggtcccctgctgcagcctacactgaatgctagtgttgaagtaatcttaaatctaatcaatggatcatcttctacataccctaattttgcccatggatgggtaaatgtaaaagatgtggcactggcacatatccttgcatatgaagttccttcagcagatggaagatattgtattgttgaaagagttgttcactacttggagcttgtcaagatcataagcaagatgtatccgaacatttcactcccatacaagtgtggagatggcgagccactttttccaacctaccaagtatggacggacaagataagaagcttgggtgtcgaactgattcctctcgagaaaactattaaagagactatcgagagcttaaaagaaaagggtttctttgcattgtga

SEQ ID NO: 14 - DNA, 1020 bp, Triticum aestivum
  source 1..1020: mol_type = other DNA
atggcagagaagcagcaacaaggatcgaacgtggaggaggggagctccagggctgcggggaaggtcgtctgcgtcaccggcgcctccgggtatatcgcgtcctggatcgtcaagctccttctcgatcgcggctacaccgtccacggcaccgtccgggacaccgctgacccagataaaacatggcacctgcgtgctcttgatggagcaaacgataggctgcagctgttcaatgcaaacttgttagaagaaggctctttcgatgctgccattgacggttgcgagtgtgtcttccatgcggcttctccagttttcttcactatcaaagatcccaaggccgagttacttgaagcagcagtcagtggaacacttgacgttctgcgttcctgcaagaaagcttctgtcaaaagagtggtcataacatcgtccatggctgctgttacctttaacgggaaaccaaggacccctgatgtaattgttgacgagacatggttttcagttccagagctatgtgaaaagcaccagcaatggtatgtcttgtccaagacccttgcagaggaggctgcgtggaagttctccaaagataatgaacttgaaatagttgtaatgcacccaacaatggtcatcggtcccctgctgcagcctacactgaatgctagtgttgaagtaatcttaaatctaatcaatgggtcatcttctacataccctaatattgcccatggatgggtaaatgtaaaagatgtggcactggcacatatccttgcatatgaagttccttcagcagatggaagatattgcattgttgaaagagttgctcactacttggagcttgtcaagatcataagcaagatgtatccgaacattccactcccatacaagcgtggagatgacgagccactttttccaacctaccaagtatcgacggacaagataagaagcttgggtgtcgaactgattcctctcgagacaactattaaagagactatcgagagcttaaaagaaaggggtttctttgaattctga

SEQ ID NO: 15 - DNA, 993 bp, Triticum aestivum
  source 1..993: mol_type = other DNA
atgagctcggggatgggcaaggtggtgtgcgtcaccggcggctcgggctacatcgcctcctggctcgtcaagctcctcctccaccgcggctacaccgtccgcgccaccgtccgggacaccgccgaccccaagaaaacgttgcacctgcaggccctggatggagccaaggataggctgcacttgttcaaagcaagcttgttagaagaaggctccttcgatgctgccatcgcgggttctgactgtgtgttccacacggcttctcccttttatcacaatgtcaaggatcccaaggctgagttacttgacccagcagtcaatggaacactcaatgttctccgttcctgcaaaaaagcctcaatcaagagagtgatcataacgtcgtccatggctgctgttgcctacaatgggaagcccagaacacctgatgtagtagttgatgagacatggttttcaagtgcagaggtctgtgaaaagaatgagcaatggtatgtgctatccaagacccttgctgaggaagctgcatggaagtttgcaaatgataatggatttgaaatagttacaataaacccagctatggtcatcggtcctctgttgcagcctacactgaatactagcgcagaggcaatattgaagtttataaatggatcatcttctacataccccaatttcagctttggatggataaatgttaaagatgttgccctggcacatattcttgcatatgaagttccttcagcaaacggaagatactgtatggttgaaagagttgctcaccactcggagattgtcaagatcatacatgaaatgtaccccaactttccagtgccagaaaagtgtgcagatgacgcaccatttgtccctatctaccaggtatcgaaggacaagataagaagtttgggcatggagctgattcccctggagacgagcctcaaggagactattgagagcttgagagagaaaggatttgttacttctgagtcgagccatctgtga

SEQ ID NO: 16 - DNA, 1011 bp, Oryza sativa
  source 1..1011: mol_type = other DNA
atgagctccgagtctgaggcggcgcccgggaccgggaagctggtgtgcgtcaccggcgcgtcggggtacatcgcgtcgtggctcgtcaggctgctcctcgcccgcggctacacggtccgcgccaccatccgggacacctctgacccaaagaaaacactacacctgcgtgccctggatggagccaacgaaagactccatttgttcgaagcaaatctgctggaagagggctctttcgatgctgcggtcaatggttgtgattgtgttttccatacggcttctcccttttaccacaatgttaaagatcctaaggctgagttacttgaccctgcagtcaaaggaacccttaatgttctaggctcctgcaagaaagcttccattagaagagttatcgtgacatcatccatggctgctgttgcctacaatgggaaaccaagaactcctgatgtagttgttgatgagacatggttttcagttccagagatttgtgaaaagcatcagcaatggtatgttctgtccaagaccctagcagaggaggctgcttggaagttttcaaaagataatggatttgaaatagttacggtgaacccagcaatggtcattggtcccctgctgcagccttcactgaatactagtgctgaagcaattctgaagctaatcaatggatcatcctcgacatatcctaatttcagctttggctggataaacgttaaggatgttgccctggcacatatccttgcatatgaagttccttcagcaaatggaagatattgcatggttgaaagagttgcgcactactcagagctcgtccaaatcattcgtgagatgtatcccaatattcctctgccagacaagtgtgcagatgacaagccatcagtgccaatctaccaggtatcaaaggaaaagataaaaagcttgggcctggaactgactcccctgcacacgagcatcaaggagaccatcgagagcttgaaagagaaaggatttgttacttttgattcaagcaacctgtaa

SEQ ID NO: 17 - DNA, 1011 bp, Zea mays; identical to SEQ ID NO: 16
  source 1..1011: mol_type = other DNA

SEQ ID NO: 18 - DNA, 1005 bp, Sorghum bicolor
  source 1..1005: mol_type = other DNA
atgagctctgcggcggcgatgacgggggcggggaaggtggtgtgcgtcaccggcgcatcagggtacatcgcgtcctggatcgtcaggctgctcctcgaccgcggctacaccgtccgcgccaccgtgcgggacaccgctgacccaaagaaaacattgcacctgactgctctggatggagccaaggataggctgcacttatttaaagctagcctgctagaagagggctcttttgacgctgcagttcatggatgtgacaccgtattccatactgcctctcccttttatcacaatgtcaaggatcctaaggctgagttacttgacccagcagttaagggaacactcaatgttcttggttcatgcaagaaagcttctattacaaaagtggttataacatcctctatggctgctgtttcctacaacgagaagccaaggactcctgaggtcacagttgacgagacatggtttactgatccacaaatttgtgaaaagactcagcaatggtatgttttgtccaagacactcgcagagcaggctgcttggaagttttcaagggataatggatttgaaattgttactataaacccagcaatggttattggtcccctgctccagcctacactaaataccagtgctgaagcaatcctgaagttaatcaatggatcatctacatatcccaattcaagctttggatgggttaatgtcaaagatgttgccctggcccatatccttgcatatgagattccctcggcaaatggaaggtattgcatggtggaaagagttgttcactactcagaactagtcaaaatcatccgcaagatgtatcctaccattcgtcttccagacaagtgtgcagatgacaagccatttgtcccaacctaccaagtatcaaaggagaagataagaagcataggcattgagttaattccagtggagacgagcgtcaaggagactatcgaaagcttgaaagagaagggatttgttagttttgactcgagcaacctgtga

SEQ ID NO: 19 - DNA, 980 bp, Medicago truncatula
  source 1..980: mol_type = other DNA
atgagcagcagcaacttgggaaatgttgtgtgtgtgactggcgcttcaggttacatcgcttcatggctcgtccgattgcttcttcatcgtggctacactgttaaagccaccgttcgcgatccaaatgatcccaagaaggttgaccacttggttaagcttgatggtgctaaggagagattgcaactgttcaaggcaaatctacttgaagaaggtgcctttgattctgttgttcaaggctgtcatggtgtctttcacactgcatctcccttttatcatgatgtcaaggatcctcgggctgaattgattgatcctgctctcaaagggactctcaatgttctcaactcatgtgctaaatcctcatcactcaaacgtgttgttttaacttcttctattgctgctgttgcttataatgggaagcctcgaactcctgatgttgttgttgatgagacttggtttacagatgctgatttctgtgctaaatcaaatctgtggtatgtggtttcaaagacattggctgaagaagctgcctggaaatttgttaaagaaaacaacatcgatatggttactattaacccagcaatggtcatagggcctctcttgcaaccagtcctaaacacaagtgctgctgcaattctaaacctgattaatggtgcacagacatttccaaatgctagttttggatgggtcaatgtgaaagatgttgcaaatgcccatattctggcgtacgagaatgcttcagcgagtggaagacattgtttagttgagagagtagcacactactccgaagttgtgaggattttacgtgaactgtacccttcgttgcaactcccagagaagtgtgcggacgataagccatatgtgcctatatatcaggtttccaaagaaaaggcgaaaagcttgggacttgaatatactcctttggaagtgagcatcaaggagactgttgaaagtttgaaagaaaagaagttcgccaaccttta

SEQ ID NO: 20 - DNA, 18 bp, synthetic construct
  source 1..18: mol_type = other DNA
gtcgggctccattgctct

SEQ ID NO: 21 - DNA, 18 bp, synthetic construct
  source 1..18: mol_type = other DNA
ccggtacccagtgacgac

SEQ ID NO: 22 - DNA, 19 bp, synthetic construct
  source 1..19: mol_type = other DNA
gcgatgtctacagtaggag

SEQ ID NO: 23 - DNA, 20 bp, synthetic construct
  source 1..20: mol_type = other DNA
gcttgagccataataccatt

SEQ ID NO: 24 - DNA, 18 bp, synthetic construct
  source 1..18: mol_type = other DNA
cactcctcgcttctcttc

SEQ ID NO: 25 - DNA, 20 bp, synthetic construct
  source 1..20: mol_type = other DNA
gattcaccattgtcactctg

SEQ ID NO: 26 - DNA, 19 bp, synthetic construct
  source 1..19: mol_type = other DNA
catcagactcaccaccttc

SEQ ID NO: 27 - DNA, 20 bp, synthetic construct
  source 1..20: mol_type = other DNA
cgtagcattcacttactcct

SEQ ID NO: 28 - DNA, 18 bp, synthetic construct
  source 1..18: mol_type = other DNA
tcgggtcaggcttttttt

SEQ ID NO: 29 - DNA, 19 bp, synthetic construct
  source 1..19: mol_type = other DNA
ggtgttcacgatagttagg

SEQ ID NO: 30 - DNA, 19 bp, synthetic construct
  source 1..19: mol_type = other DNA
tgatggatggcgttgaata

SEQ ID NO: 31 - DNA, 19 bp, synthetic construct
  source 1..19: mol_type = other DNA
cttaggcttgttggttgtc

SEQ ID NO: 32 - DNA, 22 bp, synthetic construct
  source 1..22: mol_type = other DNA
ctctaagcactttgaataaggg

SEQ ID NO: 33 - DNA, 20 bp, synthetic construct
  source 1..20: mol_type = other DNA
gcgtgtgtctctataaggaa

SEQ ID NO: 34 - DNA, 19 bp, synthetic construct
  source 1..19: mol_type = other DNA
acgaattgctcagtcagat

SEQ ID NO: 35 - DNA, 18 bp, synthetic construct
  source 1..18: mol_type = other DNA
gcggtcaggtcaagttaa

SEQ ID NO: 36 - DNA, 19 bp, synthetic construct
  source 1..19: mol_type = other DNA
cggttccatcttcttccat

SEQ ID NO: 37 - DNA, 17 bp, synthetic construct
  source 1..17: mol_type = other DNA
tcaaggcatcaagcatc

SEQ ID NO: 38 - DNA, 20 bp, synthetic construct
  source 1..20: mol_type = other DNA
tctccgaagagcatatacga

SEQ ID NO: 39 - DNA, 18 bp, synthetic construct
  source 1..18: mol_type = other DNA
gtgggtggtggattaggt

SEQ ID NO: 40 - DNA, 19 bp, synthetic construct
  source 1..19: mol_type = other DNA
cgacaatgttcatccgatt

SEQ ID NO: 41 - DNA, 18 bp, synthetic construct
  source 1..18: mol_type = other DNA
ggtacagtgttgcgagtt

SEQ ID NO: 42 - DNA, 22 bp, synthetic construct
  source 1..22: mol_type = other DNA
cactaaggaatccgatgaaatc

SEQ ID NO: 43 - DNA, 22 bp, synthetic construct
  source 1..22: mol_type = other DNA
catgacctgaaacgatgaatag

SEQ ID NO: 44 - DNA, 19 bp, synthetic construct
  source 1..19: mol_type = other DNA
ctcactattcagccagaca

SEQ ID NO: 45 - DNA, 20 bp, synthetic construct
  source 1..20: mol_type = other DNA
attgacaggagacatgacat

SEQ ID NO: 46 - DNA, 19 bp, synthetic construct
  source 1..19: mol_type = other DNA
atcactgctgctcagatag

SEQ ID NO: 47 - DNA, 18 bp, synthetic construct
  source 1..18: mol_type = other DNA
tgcgtcttcttcaccatg

SEQ ID NO: 48 - DNA, 20 bp, synthetic construct
  source 1..20: mol_type = other DNA
ggtttagaatttgtccaggt

SEQ ID NO: 49 - DNA, 20 bp, synthetic construct
  source 1..20: mol_type = other DNA
gttgggcgtttcaataaatg

SEQ ID NO: 50 - DNA, 19 bp, synthetic construct
  source 1..19: mol_type = other DNA
atataaaccgtccctccct

SEQ ID NO: 51 - DNA, 19 bp, synthetic construct
  source 1..19: mol_type = other DNA
ccatcgttgatgatgagtg

SEQ ID NO: 52 - DNA, 20 bp, synthetic construct
  source 1..20: mol_type = other DNA
gaaggcttctgtcagaagag

SEQ ID NO: 53 - DNA, 20 bp, synthetic construct
  source 1..20: mol_type = other DNA
agtggtcataacatcgtcta

SEQ ID NO: 54 - DNA, 20 bp, synthetic construct
  source 1..20: mol_type = other DNA
atggcttctgctctcttcac

SEQ ID NO: 55 - DNA, 20 bp, synthetic construct
  source 1..20: mol_type = other DNA
atgtaattgttgatgaaaca

SEQ ID NO: 56 - DNA, 20 bp, synthetic construct
  source 1..20: mol_type = other DNA
gtttcatcaacaattacatc

SEQ ID NO: 57 - DNA, 20 bp, synthetic construct
  source 1..20: mol_type = other DNA
atcaggggttcttggtttcc

SEQ ID NO: 58 - DNA, 20 bp, synthetic construct
  source 1..20: mol_type = other DNA
ggacaccgctgacccaaata

SEQ ID NO: 59 - DNA, 20 bp, synthetic construct
  source 1..20: mol_type = other DNA
tgcaaggatatgtgccaggg

SEQ ID NO: 60 - DNA, 20 bp, synthetic construct
  source 1..20: mol_type = other DNA
agagctatgtgaaaagcacc

SEQ ID NO: 61 - DNA, 20 bp, synthetic construct
  source 1..20: mol_type = other DNA
gttaaaggtaacagcagcca

SEQ ID NO: 62 - DNA, 20 bp, synthetic construct
  source 1..20: mol_type = other DNA
ggtccttggtttcccgttaa

SEQ ID NO: 63 - DNA, 20 bp, synthetic construct
  source 1..20: mol_type = other DNA
agtggtcataacatcgtcca

SEQ ID NO: 64 - DNA, 20 bp, synthetic construct
  source 1..20: mol_type = other DNA
atgtaattgttgacgagaca

SEQ ID NO: 65 - DNA, 20 bp, synthetic construct
  source 1..20: mol_type = other DNA
gaaagcttctgtcaaaagag

SEQ ID NO: 66 - DNA, 20 bp, synthetic construct
  source 1..20: mol_type = other DNA
gtccaagacccttgcagagg

SEQ ID NO: 67 - DNA, 20 bp, synthetic construct
  source 1..20: mol_type = other DNA
acaattacatcaggggtcct

SEQ ID NO: 68 - DNA, 20 bp, synthetic construct
  source 1..20: mol_type = other DNA
gtctcgtcaacaattacatc

SEQ ID NO: 69 - DNA, 20 bp, synthetic construct
  source 1..20: mol_type = other DNA
agcctcctctgcaagggtct

SEQ ID NO: 70 - DNA, 20 bp, synthetic construct
  source 1..20: mol_type = other DNA
atggctgctgttacctttaa

SEQ ID NO: 71 - DNA, 24 bp, synthetic construct
  source 1..24: mol_type = other DNA
tggcagtggtcataacatcgtcca

SEQ ID NO: 72 - DNA, 24 bp, synthetic construct
  source 1..24: mol_type = other DNA
aaactggacgatgttatgaccact

SEQ ID NO: 73 - DNA, 24 bp, synthetic construct
  source 1..24: mol_type = other DNA
gtgttggctgctgttacctttaac

SEQ ID NO: 74 - DNA, 24 bp, synthetic construct
  source 1..24: mol_type = other DNA
aaacgttaaaggtaacagcagcca

SEQ ID NO: 75 - DNA, 24 bp, synthetic construct
  source 1..24: mol_type = other DNA
tagcaggacccctgatgtaattgt

SEQ ID NO: 76 - DNA, 24 bp, synthetic construct
  source 1..24: mol_type = other DNA
aaacacaattacatcaggggtcct

SEQ ID NO: 77 - DNA, 20 bp, synthetic construct
  source 1..20: mol_type = other DNA
aacgataggctgcagctgtt

SEQ ID NO: 78 - DNA, 20 bp, synthetic construct
  source 1..20: mol_type = other DNA
ctcgtcatctccacgcttgt

SEQ ID NO: 79 - protein, 324 aa, Triticum aestivum
MSSGMGKVVCVTGASGYIASWLVKLLLHRGYTVRATVRDTADPKKTLHLQALDGAKDRLHLFKASLLEEGSFDAAVAGCDCVFHTASPFYHNVKDPKAELLDPAVNGTLNVLRSCKKASIKRVIITSSMAAVAYNGKPRTPDVVVDETWFSSAEQWYVLSKTLAEEAAWKFANDNGFEIVTINPAMVIGPLLQPTLNTSAEAILKFINGSSSTYPNFSFGWVNVKDVALSHILAYEVPSANGRYCMVERVAHHSEIVKIIHEMYPNFPVPDKCADDAPFVPIYQVSKDKIRSLGMELIPLETSLKETIESLREKGFVTSESSHL

SEQ ID NO: 80 - protein, 330 aa, Triticum aestivum
MSSGMGKVVCVTGASGYIASWLVKLLLHRGYTVRATVRDTADPKKTLHLQALDGAKDRLHLFKASLLEEGSFDAAVAGCDCVFHTASPFYHNVKDPKAELLDPAVNGTLNVLRSCKKASIKRVIITSSMAAVAYNGKPRTPDVVVDETWFSSAEVCEKNEQWYVLSKTLAEEAAWKFANDNGFEIVTINPAMVIGPLLQPTLNTSAEAILKFINGSSSTYPNFSFGWVNVKDVALSHILAYEVPSANGRYCMVERVAHHSEIVKIIHEMYPNFPVPDKCADDAPFVPIYQVSKDKIRSLGMELIPLETSLKETIESLREKGFVTSESSHL

SEQ ID NO: 81 - protein, 330 aa, Triticum aestivum
MSSGMGKVVCVTGASGYIASWLVKLLLHRGYTVRATVRDTADPKKTLHLQALDGAKDRLHLFKASLLEEGSFDAAIAGSDCVFHTASPFYHNVKDPKAELLDPAVNGTLNVLRSCKKASIKRVIITSSMAAVAYNGKPRTPDVVVDETWFSSAEVCEKNEQWYVLSKTLAEEAAWKFANDNGFEIVTINPAMVIGPLLQPTLNTSAEAILKFINGSSSTYPNFSFGWINVKDVALAHILAYEVPSANGRYCMVERVAHHSEIVKIIHEMYPKFPVPDKCADDAPFVPIYQVSKDKIRSLGMELIPLETSLKETIESLREKGFVTSESSHL

SEQ ID NO: 82 - protein, 330 aa, Triticum aestivum
MSSGMGKVVCVTGGLGYIASWLVKLLLHRGYTVCATVRDTADPKKTLHLQALDGAKDRLHLFKASLLEEGSFDAAIAGSDCVFHTASPFYHNVKDPKAELLDPAVNGTLNVLRSCKKASIKRVIITSSMAAVAYNGKPRTPDVVVDETWFSSAEVCEKNEQWYVLSKTLAEEAAWKFANDNGFEIVTINPAMVIGPLLQPTLNTSAEAILKFINGSSSTYPNFSFGWINVKDVALAHILAYEVPSANGRYCMVERVAHHSEIVKIIHEMYPNFPVPEKCADDAPFVPIYQVSKDKIRSLGMELIPLETSLKETIESLREKGFVTSESSHL

SEQ ID NO: 83 - protein, 330 aa, Triticum aestivum
MSSGMGKVVCVTGGSGYIASWLVKLLLHRGYTVRATVRDTADPKKTLHLQALDGAKDRLHLFKASLLEEGSFDAAIAGSDCVFHTASPFYHNVKDPKAELLDPAVNGTLNVLRSCKKASIKRVIITSSMAAVAYNGKPRTPDVVVDETWFSSAEVCEKNEQWYVLSKTLAEEAAWKFANDNGFEIVTINPAMVIGPLLQPTLNTSAEAILKFINGSSSTYPNFSFGWINVKDVALAHILAYEVPSANGRYCMVERVAHHSEIVKIIHEMYPNFPVPEKCADDAPFVPIYQVSKDKIRSLGMELIPLETSLKETIESLREKGFVTSESSHL

SEQ ID NO: 84 - DNA, 975 bp, Triticum aestivum
  source 1..975: mol_type = other DNA
atgagctccgggatggggaaggtggtgtgcgtcaccggcgcctcgggctacatcgcctcctggctcgtcaagctcctccttcaccgcggctacaccgtccgcgccaccgtcagggacaccgccgaccccaagaaaacattgcacctgcaggccctggatggagccaaggacaggctccacttgttcaaagcaagcttgttggaagaaggctctttcgatgctgccgttgccggctgcgattgtgtcttccacacggcctctcccttttatcacaatgtcaaggatcccaaggctgagttacttgacccagctgtcaatggaacactcaatgttctccgttcctgcaagaaagcctctatcaagagagtgatcataacgtcgtccatggctgctgttgcctacaatgggaagccaagaactcctgatgtagtggttgatgagacatggttttcaagtgcagagcaatggtatgtgctatccaagacccttgctgaggaagctgcatggaagttcgcaaatgataatggatttgaaatagttacaataaacccagcaatggtcatcggtcccctgttgcagcctacactgaatactagcgcagaagcaatattgaagtttataaatggatcatcttctacataccccaatttcagctttggctgggtaaatgttaaagatgttgccctgtcacatatccttgcatatgaagttccttcggcaaacggaagatactgtatggttgaaagagttgctcaccactcggagattgtcaagatcatacacgaaatgtaccccaattttccagtgccagacaagtgtgcagatgacgcaccatttgtccctatctaccaggtatcgaaggacaagataagaagtttgggtatggagctgattcccctggagacgagcctcaaggagactatcgagagcttgagagagaaaggatttgttacttctgagtcaagccatctatga

SEQ ID NO: 85 - DNA, 993 bp, Triticum aestivum
  source 1..993: mol_type = other DNA
atgagctccgggatggggaaggtggtgtgcgtcaccggcgcctcgggctacatcgcctcctggctcgtcaagctcctccttcaccgcggctacaccgtccgcgccaccgtcagggacaccgccgaccccaagaaaacattgcacctgcaggccctggatggagccaaggacaggctccacttgttcaaagcaagcttgttggaagaaggctctttcgatgctgccgttgccggctgcgattgtgtcttccacacggcctctcccttttatcacaatgtcaaggatcccaaggctgagttacttgacccagctgtcaatggaacactcaatgttctccgttcctgcaagaaagcctctatcaagagagtgatcataacgtcgtccatggctgctgttgcctacaatgggaagccaagaactcctgatgtagtggttgatgagacatggttttcaagtgcagaggtctgtgaaaagaatgagcaatggtatgtgctatccaagacccttgctgaggaagctgcatggaagttcgcaaatgataatggatttgaaatagttacaataaacccagcaatggtcatcggtcccctgttgcagcctacactgaatactagcgcagaagcaatattgaagtttataaatggatcatcttctacataccccaatttcagctttggctgggtaaatgttaaagatgttgccctgtcacatatccttgcatatgaagttccttcggcaaacggaagatactgtatggttgaaagagttgctcaccactcggagattgtcaagatcatacacgaaatgtaccccaattttccagtgccagacaagtgtgcagatgacgcaccatttgtccctatctaccaggtatcgaaggacaagataagaagtttgggtatggagctgattcccctggagacgagcctcaaggagactatcgagagcttgagagagaaaggatttgttacttctgagtcaagccatctatga

SEQ ID NO: 86 - DNA, 993 bp, Triticum aestivum
  source 1..993: mol_type = other DNA
atgagctccgggatggggaaggtggtgtgcgtcaccggcgcctcgggctacatcgcctcctggctcgtcaagctcctcctccaccgcggctacaccgtccgcgccaccgtcagggacaccgccgaccccaagaaaacattgcacctgcaggccctggatggagccaaggacaggctgcacttgttcaaagcaagcttattggaagaaggctctttcgatgcggccattgccggttctgattgtgtcttccacacggcttctcccttttatcacaatgtcaaggatcccaaggctgagttacttgacccagctgtcaatggaacactcaatgttctccgttcctgcaagaaagcctctatcaagagagtgatcataacatcgtccatggctgcggttgcctacaatgggaagccaagaactcctgatgtagtagttgatgagacatggttttcaagtgcagaggtctgtgaaaagaatgagcaatggtatgtgctatccaagacccttgctgaggaagctgcatggaagttcgcaaatgataatggatttgaaatagttacaataaacccagcaatggtcatcggtcccctgttgcagcctacactgaatactagcgcagaagcaatattgaagtttataaatggatcatcttctacataccccaatttcagctttggctggataaatgttaaagatgttgccctggcacatatccttgcatatgaagttccttcggcaaacggaagatactgtatggttgaaagagttgctcaccactcggagattgtcaagatcatacacgaaatgtaccccaaatttccagtgccagacaagtgtgcagatgacgcaccatttgtccctatctaccaggtatcgaaggacaagataagaagtttgggtatggagctgattcccctggagacgagcctcaaggaaactatcgagagcttgagagagaaaggatttgttacttctgagtcaagccatctatga

SEQ ID NO: 87 - DNA, 993 bp, Triticum aestivum
  source 1..993: mol_type = other DNA
atgagctccgggatggggaaggtggtgtgcgtcaccggcggcttgggctacatcgcctcctggctcgtcaagctcctcctccaccgcggctacaccgtctgtgccaccgtcagggacaccgccgaccccaagaaaacgttgcacctgcaggccctggatggagccaaggataggctgcacttgttcaaagcaagcttgttagaagaaggctccttcgatgctgccatcgcgggttctgactgtgtgttccacacggcttctcccttttatcacaatgtcaaggatcccaaggctgagttacttgacccagcagtcaatggaacactcaatgttctccgttcctgcaaaaaagcctcaatcaagagagtgatcataacgtcgtccatggctgctgttgcctacaatgggaagcccagaacacctgatgtagtagttgatgagacatggttttcaagtgcagaggtctgtgaaaagaatgagcaatggtatgtgctatccaagacccttgctgaggaagctgcatggaagtttgcaaatgataatggatttgaaatagttacaataaacccagctatggtcatcggtcctctgttgcagcctacactgaatactagcgcagaggcaatattgaagtttataaatggatcatcttctacataccccaatttcagctttggatggataaatgttaaagatgttgccctggcacatattcttgcatatgaagttccttcagcaaacggaagatactgtatggttgaaagagttgctcaccactcggagattgtcaagatcatacatgaaatgtaccccaactttccagtgccagaaaagtgtgcagatgacgcaccatttgtccctatctaccaggtatcgaaggacaagataagaagtttgggcatggagctgattcccctggagacgagcctcaaggagactattgagagcttgagagagaaaggatttgttacttctgagtcgagccatctgtga

SEQ ID NO: 88 - DNA, 993 bp, Triticum aestivum; identical to SEQ ID NO: 15
  source 1..993: mol_type = other DNA